(19) United States
(12) Patent Application Publication
(10) Pub. No.: US 2016/0331326 A1
(43) Pub. Date: Nov. 17, 2016

(54) FLEXIBLE NEURAL STRIP ELECTRODES, FLEXIBLE NEURAL RIBBON ELECTRODES AND COMPARTMENT BASED EMBEDDED NERVE TISSUE ELECTRODE INTERFACES FOR PERIPHERAL NERVES

(71) Applicant: National University of Singapore, Singapore (SG)

(72) Inventors: Zhuolin XIANG, Singapore (SG); Shih-Cheng YEN, Singapore (SG); Chengkuo LEE, Singapore (SG); Sanghoon LEE, Singapore (SG); Nitish V. THAKOR, Singapore (SG); Amitabha LAHIRI, Singapore (SG); Faith Ann BAZLEY, Singapore (SG); Ashwati VIPIN, Singapore (SG); Ignacio Delgado MARTINEZ, Singapore (SG); Angelo Homayoun ALL, Singapore (SG); Yen Xian PEH, Singapore (SG)

(21) Appl. No.: 15/043,688

(22) Filed: Feb. 15, 2016

Related U.S. Application Data
(60) Provisional application No. 62/176,387, filed on Feb. 13, 2015.

(51) Int. Cl. A61B 5/00; A61N 1/05; A61B 5/04

(52) U.S. Cl. CPC A61B 5/6877; A61B 5/04001; A61N 1/0551; A61N 1/0556; A61B 2562/164; A61B 2562/166

(57) ABSTRACT

Embodiments in accordance with the present disclosure are directed to non-invasive or essentially non-invasive electrode structures, assemblies, and devices for sensing neural signals carried or produced by peripheral nerves, and/or applying stimulation signals to peripheral nerves. Electrode structures, assemblies, and devices in accordance with embodiments of the present disclosure include (a) flexible epineural strip electrode structures having one or more elongate electrode-carrying strips that can be adhered (e.g., glued) and/or sutured to a peripheral nerve; (b) flexible elongate ribbon electrode structures, which can be spirally wound about portions of a peripheral nerve's length such that microneedle and/or disc or stud type electrodes carried by the ribbon electrode structure are disposed in a helical arrangement about the peripheral nerve; and (c) an embedded nerve tissue—electrode interface having a tubular compartment containing adipose tissue that supports axonal tissue ingrowth and interfacing of ingrown axonal tissue with electrode microwires in the compartment.

### CROSS REFERENCE TO RELATED APPLICATIONS

This application claims priority to the following patent application: (1) U.S. Patent Application 62/176,387 filed Feb. 13, 2015; the above cited application is hereby incorporated by reference herein as if fully set forth in its entirety.

### TECHNICAL FIELD

Aspects of the present disclosure relate to particular types of neural electrode structures by which neural signals can be sensed from and/or stimulation signals applied to peripheral nerves. Such electrode structures include: flexible epineural strip electrodes; flexible neural ribbon electrodes; and an embedded nerve tissue—electrode interface having a tubular compartment into which axonal tissue ingrowth and interfacing with electrode signal transfer structures or materials (e.g., microwires) can occur.

### BACKGROUND

A growing field of electrophysiology research involves finding a reliable approach for recording tiny neural signals that travel through peripheral nerves. For decades, scientists have been able to accurately detect neural spikes from the brain (e.g., the cortex), but reliably acquiring neural signals directly from peripheral nerves has proven to be a much harder challenge. This is due to the physiological, anatomical, and electrical characteristics of peripheral nerves and the environment in which they reside. In particular, i) axons are surrounded by insulating myelin, and bundled into fascicles which are further surrounded by dense protective outer layers, known as the perineurium and epineurium; ii) axons are bundled densely inside the nerve so that it is difficult to distinguish between the neural signals from neighboring axons or even fascicles; iii) any recorded peripheral nerve signals are inherently several orders of magnitude smaller than brain or cortex neural signals (e.g., neural signals recorded from peripheral nerves can have a magnitude of approximately 8-10 μV or less); and iv) additional interfering noise sources are present, including muscle and movement artifacts, which corrupt peripheral nerve signals. There is currently no suitable peripheral nerve electrode design that provides a stable nerve tissue-electrode interface that can reliably pick up neuroelectric signals on a long-term basis.

Various kinds of peripheral nerve electrode designs have been developed, such as neural cuff electrodes, longitudinal intrafasicular electrodes (LIFE), transverse intrafasicular multichannel electrodes (TIME), and flat nerve interface electrodes (FINE), among others (e.g., regenerative/sieve electrodes). Neural cuff electrodes have been widely used chronically in different clinical applications owing to their low invasiveness. In addition, snug-fitting nerve cuffs have been approved to reduce the stimulus charge injection or to obtain a high signal-to-noise ratio (SNR) for neural recording. However, delicate nerve tissue can be damaged by the presence of the cuff due to the physical properties of the cuff electrode, which is typically much stiffer than the nerve. Also, chronic implantation of snug cuff electrodes modifies the nerve shape and produces a loss of large nerve fibers as a result of compression of the nerve by the cuff electrodes. Moreover, cuff electrodes have a large footprint, and can only be applied to main nerve bundles having large diameters, and cannot be attached to small nerve bundles or branches. In addition, nerve cuffs, which are typically made with silicone tubes with a longitudinal slit, have to be held open manually during nerve placement. This inexact process is technically difficult and poses a significant risk of nerve damage when installing such electrodes onto small diameter nerves. Therefore, alternative peripheral nerve electrode designs are needed.

### SUMMARY

In accordance with an embodiment of the present disclosure, a flexible epineural strip electrode for a peripheral nerve includes: a single flexible substrate (e.g., made of polyimide or parylene) having a nerve interface portion and an electronics interface portion that extends away from the nerve interface portion, wherein the nerve interface portion includes an inner surface configured for direct placement upon the epineurium of the peripheral nerve, wherein the inner surface carries a set of exposed electrodes configured for contacting the epineurium of the peripheral nerve, wherein the electronics interface portion carries at least one set of electrical pads to which an electrical device distinct from the flexible substrate can be electrically coupled, and wherein the nerve interface portion and the electronics interface portion carry integrated circuit wiring by which the set of electrical contacts is electrically coupled to at least one set of electrical pads.

The nerve interface portion can include a plurality of suture apertures formed therein by which the nerve interface portion is suturable to the peripheral nerve, another anatomical structure, or itself.

The flexible epineural strip electrode further can further an integrated circuit chip, a flexible printed circuit (FPC), or a flexible flat cable (FFC) bonded to the at least one set of electrical pads, wherein the integrated circuit chip, the FPC, or the FFC corresponds to a neural amplifier or a neural stimulator.

The nerve interface portion includes at least one flexible elongate strip. For instance, the nerve interface portion can include a plurality of flexible elongate strips disposed in a parallel arrangement with respect to each other, wherein each flexible elongate strip includes an inner surface configured for direct placement on the epineurium of the peripheral nerve, and wherein the inner surface of each flexible elongate strip carries a plurality of exposed electrodes configured for contacting the epineurium of the peripheral nerve.

In accordance with an aspect of the present disclosure, a flexible epineural strip electrode for a peripheral nerve includes: a single flexible substrate (e.g., made of polyimide or parylene) having a front side configured for facing away from the epineurium of the peripheral nerve and a back side configured for direct placement upon the epineurium of the peripheral nerve, wherein the front side carries a neural amplifier or neural stimulator, and wherein the back side carries a plurality of exposed electrodes configured for contacting the epineurium of the peripheral nerve. The flexible substrate can include a plurality of suture apertures formed therein by which the flexible substrate is suturable to the peripheral nerve, another anatomical structure, or itself.

The single flexible substrate can include a plurality of flexible strips, each flexible strip having a front side configured for facing away from the epineurium of the peripheral nerve and a back side configured for direct placement upon the epineurium of the peripheral nerve, wherein the plurality of flexible strips includes a first flexible strip that carries the neural amplifier or neural stimulator on its front side and which further carries a first set of exposed electrodes on its back side, and a second flexible strip that carries a second set of exposed electrodes on its back side. Each flexible strip can be structurally coupled to an adjacent flexible strip by way of a set of arm members, and each flexible strip includes suture apertures formed therein by which the flexible strip is sutrable to the peripheral nerve, another anatomical structure, itself, or another flexible strip.

In accordance with an aspect of the present disclosure, a flexible neural ribbon electrode for a peripheral nerve includes a single flexible substrate (e.g., made of polyimide or parylene) having: an elongate ribbon section having an outer surface configured for facing away from the epineurium of the peripheral nerve and an inner surface configured for facing toward the epineurium of the peripheral nerve, wherein the elongate ribbon section is spirally windable about the epineurium along a portion of a length of the peripheral nerve; a plurality of electrodes disposed along and projecting from the inner surface of the elongate ribbon section; and a first end portion providing a connection pad structure having a plurality of electrical pads to which an electronic device distinct from the flexible neural ribbon electrode is electrically couplable or bondable.

The flexible neural ribbon electrode of claim further includes a second end portion, wherein the elongate ribbon section extends between the first end portion and the second end portion. The first end portion and the second end portion can include suture apertures formed therein by which the first end portion and the second end portion, respectively, are suturable to the peripheral nerve, one or more other anatomical structures, and/or themselves.

The plurality of electrodes can include microneedle electrodes configured for penetrating the epineurium, and/or stud type electrodes configured for directly residing upon the epineurium surface.

The flexible neural ribbon electrode can also include a reference electrode carried by an inner surface of the flexible neural electrode.

In accordance with an aspect of the present disclosure, an embedded nerve tissue—electrode interface structure includes: a biocompatible tubular compartment (e.g., made of silicone) having a first segment, a second segment disposed opposite to the first segment, and an intermediary region that extends between the first segment and the second segment; a microelectrode device having a set of electrical signal transfer structures disposed at the first segment of the tubular compartment, which extend into the intermediary region; an aperture within the second segment configured for receiving a severed peripheral nerve such that a terminal end of the peripheral nerve is disposed in the intermediary region and faces the set of electrical signal transfer structures; and a medium carried within the intermediary region that promotes axonal cellular growth.

The medium can include at least one of autologous adipose tissue, glial cells, Schwann cells, stem cells, and a nerve growth stimulant. The set of electrical signal transfer structures can include an array of microwires.

After a tissue growth period, the embedded nerve tissue—electrode interface can further include a self-organized nerve interface cone comprising fibro-collagenous axonal tissue that surrounds and physically contacts the set of electrical signal transfer structures.

### BRIEF DESCRIPTION OF THE DRAWINGS

FIGS. 1-5C are schematic illustrations of representative flexible epineural strip electrodes or electrode assemblies in accordance with particular embodiments of the disclosure.

FIGS. 6(a)-6(i) illustrate aspects of a representative process by which a flexible epineural strip electrode was fabricated in accordance with an embodiment of the present disclosure.

FIG. 7(a) is an SEM image showing an electrode surface prior to multi-wall carbon nanotube (MWCNT) coating; and FIGS. 7(b) and 7(c) are SEM images showing MWCNT coated electrode surfaces.

FIGS. 8A and 8B are graphs illustrating electrode interfacial impedance characterization for electrodes with and without gold-carbon nanotube (Au-CNT) coatings in accordance with an embodiment of the present disclosure.

FIG. 9A is a photograph of an as-fabricated flexible epineural strip electrode, and FIG. 9B schematically illustrates how this representative flexible epineural strip electrode can be positioned upon a peripheral nerve in accordance with an embodiment of the present disclosure.

FIG. 9C schematically illustrates an experimental study setup in which the as-fabricated flexible epineural strip electrode of FIG. 9A was positioned on a proximal segment of a rat sciatic nerve; and FIG. 9D is a photograph showing this as-fabricated flexible epineural strip electrode sutured to the rat sciatic nerve.

FIGS. 10(a) and 10(b) correspond to recorded compound nerve action potentials (CNAPs) recorded by way of the experimental study setup of FIGS. 9C and 9D.

FIG. 11A shows another as-fabricated flexible epineural strip electrode; and FIG. 11B schematically illustrates a representative manner in which this as-fabricated flexible epineural strip electrode can be positioned on a peripheral nerve in accordance with an embodiment of the present disclosure.

FIG. 11C schematically illustrates an experimental study setup in which one as-fabricated flexible epineural strip electrode of FIG. 11A was positioned on a proximal segment of a rat sciatic nerve, and two additional as-fabricated flexible epineural strip electrodes of FIG. 11A were positioned on peroneal and tibial branches of the rat sciatic nerve; and FIG. 11D is a photograph showing the three as-fabricated flexible epineural strip electrodes of FIG. 11A positioned on the proximal segment of the rat sciatic nerve, the peroneal branch of the rat sciatic nerve, and the tibial branch of the rat sciatic nerve. FIGS. 12A and 12B respectively show recorded CNAPs corresponding to the experimental setup of FIGS. 11C and 11D.

FIG. 13 is a schematic illustration of a representative flexible neural ribbon electrode in accordance with an embodiment of the present disclosure; and FIG. 14 is schematic illustrations showing portions of this flexible neural ribbon electrode positioned on a peripheral nerve.

FIGS. 15(a)-(j) illustrate aspects of a representative process by which a flexible neural ribbon electrode that carries microneedle electrodes can be fabricated in accordance with an embodiment of the present disclosure.

FIGS. 16A and 16B show an optical image and a SEM image, respectively, of as-fabricated microneedle electrodes in accordance with an embodiment of the present disclosure.

FIG. 17 is a schematic illustration of a representative flexible neural ribbon electrode in accordance with another embodiment of the present disclosure; and FIG. 18 is schematic illustrations showing portions of this flexible neural ribbon electrode positioned on a peripheral nerve.

FIG. 19(a) is an SEM image of Au particles and CNTs deposited on a stud-type electrode; and FIGS. 19(b) and 19(c) are SEM images showing a Au coated electrode and a CNT coated electrode, respectively.

FIGS. 20A and 20C illustrate the implantation of flexible neural ribbon electrodes on three terminal branches of a rat sciatic nerve having different diameters, namely, the peroneal nerve, tibial nerve, and sural nerve; and FIG. 20B illustrates experimental setup details for evoking and recording compound action potentials (CAPs).

FIGS. 21(a)-21(d) show recorded signals under 0.6 mA stimulation from 4 different neural ribbon electrodes corresponding to FIGS. 20A-20C.

FIG. 22(a) shows CAP recording amplitude from the rat sciatic nerve, and FIGS. 22(b)-(d) show recording amplitudes from the rat peroneal nerve, tibial nerve, and sural nerve, respectively, corresponding to FIGS. 20A-20C.

FIG. 23 shows neural signal latency measurements corresponding to FIGS. 20A-20C.

FIG. 24 is a schematic illustration of a compartment-based embedded nerve tissue—electrode interface structure in accordance with an embodiment of the present disclosure.

FIGS. 25A and 25B are images showing an experimental setup at implantation and extraction of an as-fabricated compartment-based embedded nerve tissue—electrode interface structure corresponding to FIG. 24.

FIG. 26 is a graph showing neural signal recording results obtained using the compartment-based embedded nerve tissue—electrode interface of FIGS. 25A-25B.

FIGS. 27A and 27B are plots showing values of peak amplitude and nerve conduction velocity, respectively, on the Y-axis, recorded at each of 5 sensing electrodes recorded at increasing current intensity represented on the X-axis.

FIG. 28 is a photograph showing an explanted compartment-based embedded nerve tissue—electrode interface structure having a split therein to aid examination of internal structures.

FIG. 29 is a photograph showing encasement of microelectrodes within a well-defined nerve interface cone structure.

### DETAILED DESCRIPTION

In the present disclosure, depiction of a given element or consideration or use of a particular element number in a particular FIG. or a reference thereto in corresponding descriptive material can encompass the same, an equivalent, or an analogous element or element number identified in another FIG. or descriptive material associated therewith. The use of “/” in a FIG. or associated text is understood to mean “and/or” unless otherwise indicated. The recitation of a particular numerical value or value range herein is understood to include or be a recitation of an approximate numerical value or value range.

As used herein, the term “set” corresponds to or is defined as a non-empty finite organization of elements that mathematically exhibits a cardinality of at least 1 (i.e., a set as defined herein can correspond to a unit, singlet, or single element set, or a multiple element set), in accordance with known mathematical definitions (for instance, in a manner corresponding to that described in An Introduction to Mathematical Reasoning: Numbers, Sets, and Functions, “Chapter 11: Properties of Finite Sets” (e.g., as indicated on p. 140), by Peter J. Eccles, Cambridge University Press (1998)). In general, an element of a set can include or be a system, an apparatus, a device, a structure, an object, a process, a physical parameter, or a value depending upon the type of set under consideration.

Embodiments in accordance with the present disclosure are directed to particular types of electrode structures, assemblies, and devices for sensing neural signals carried or produced by peripheral nerves, and/or applying stimulation signals (e.g., extrinsic electrical stimulation signals) to peripheral nerves. Such electrode structures, assemblies, and devices can be considered non-invasive, essentially non-invasive, or nearly non-invasive with respect to the epineurium or epineural sheath. Electrode structures, assembly, and devices in accordance with embodiments of the present disclosure are suitable for application to a wide or very wide range of sizes of peripheral nerves (e.g., including small or very small peripheral nerves having a diameter of approximately 50-250 microns), and minimize or avoid nerve tissue damage, nerve compression, nerve shape distortion, and nerve blood flow constriction. Electrode structures, assemblies, and devices in accordance with embodiments of the present disclosure include (a) flexible epineural strip electrode structures having one or more elongate (d) electrode-carrying strips that can be adhered (e.g., glued) and/or sutured to a peripheral nerve; (b) flexible elongated ribbon electrode structures, which can be spirally wound about portions of a peripheral nerve's length such that microneedle and/or disc or stud type electrodes carried by the ribbon electrode structure are positioned in a helical arrangement about the peripheral nerve; and (c) an embedded nerve tissue—electrode interface having a tubular compartment that supports axonal tissue ingrowth and interfacing of ingrown axonal tissue with electrode microwires in the compartment.

### Flexible Epineural Strip Electrode Structures

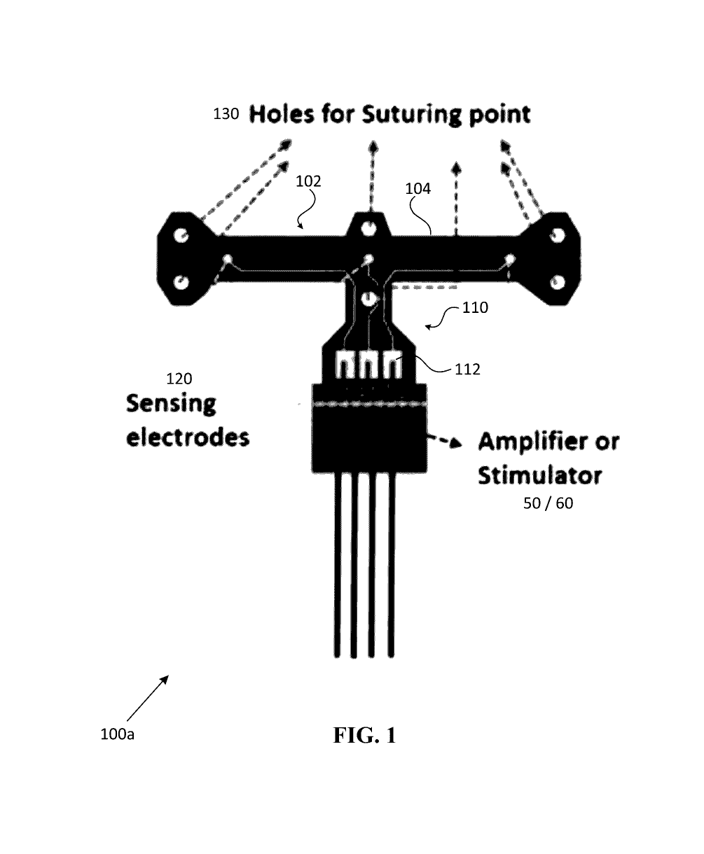

FIG. 1 is a schematic illustration of a representative flexible epineural strip electrode or electrode assembly 100a in accordance with an embodiment of the disclosure. In an embodiment, the epineural strip electrode 100a is formed of a single, unitary, or unified flexible substrate having at least one nerve interface portion 102, and an electronics interface portion 110. The nerve interface portion 102 is configured for direct placement or positioning on or along portions of a peripheral nerve's epineurium or epineural sheath, and carries a set of at least 2 electrodes or electrical contacts 120 capable of sensing neuroelectric signals from and/or applying stimulation signals to the peripheral nerve on which the nerve interface portion 102 is placed or resides. More particularly, the nerve interface portion 102 includes an outer surface that faces away from the peripheral nerve's epineurium, and an inner surface that faces towards the peripheral nerve's epineurium when the flexible epineural strip electrode 100a is positioned or mounted on or secured to the peripheral nerve. The electrodes 102 are exposed on the inner surface of the nerve interface portion 102, such that when the nerve interface portion 102 is placed or resides on the peripheral nerve, the electrodes 120 directly lie or reside along and contact portions of the epineurium. In the embodiment shown in FIG. 1, the nerve interface portion 102 includes or is formed as a flexible elongate strip 104 carrying three electrodes 120 along its length, namely, two outer lateral electrodes 120 disposed proximate to lateral borders or edges of the strip 104, and a central inner electrode 120 disposed at or near a mid-point or center point of the flexible elongate strip 104. Individuals having ordinary skill in the art will clearly understand that the flexible elongate strip 104 can carry fewer or more than three electrodes 120 depending upon embodiment details.

In various embodiments, the nerve interface portion 102 includes a plurality of apertures, holes, openings, or windows formed therein, by which the nerve interface portion 102 can be sutured to an underlying peripheral nerve (e.g., through the epineurium), one or more other anatomical structures, and/or itself. In the embodiment shown in FIG. 1, the nerve interface portion 102 includes a total of six suture apertures 130, namely, two suture apertures 130 disposed near each outer lateral border of the strip 104; and two suture apertures 130 disposed near the mid-point or center of the strip 104. Other embodiments can include more or fewer suture apertures 130. The flexible epineural strip electrode 100a can additionally or alternatively be adhered to a peripheral nerve by way of a biocompatible adhesive (e.g., tissue glue or fibrin glue, or another conventional type of flexible adhesive) and/or gel (e.g., a bio-gel).

The electronics interface portion 110 can be a portion of the flexible substrate that extends or projects away from the nerve interface portion 102 in a predetermined direction at a predetermined spatial region or section thereof. For instance, the electronics interface portion 110 can extend in a non-parallel direction (e.g., a perpendicular or approximately perpendicular direction) away from a mid-point or center point of an elongate nerve interface portion 102. The electronics interface portion 110 includes carries at least one set of electrical pads 112 to which the electrodes 120 carried by the nerve interface portion 102 are electrically coupled or linked by way of integrated circuit wiring that runs along the nerve interface portion 102 and the electronics interface portion 110. Depending upon embodiment details, the electrical pads 112 can be carried on an outer surface of the electronics interface portion 110 that faces away from the epineurium of the peripheral nerve, and/or an opposite inner surface of the electronics interface portion 110. The electrical pads 112 provide a physical interface by which the flexible epineural strip electrode 100a can be electrically coupled to other electronic circuitry (i.e., electronic circuitry other than the flexible epineural strip electrode 100a itself), such as an integrated circuit chip, a flexible printed circuit (FPC), or a flexible flat cable (FFC) corresponding to a neural amplifier 50 and/or a neural stimulator 60.

Outer or outward facing portions or surfaces of the nerve interface portion 102 that face away from the epineurium, as well as inner or inward facing and/or outer or outward facing portions or surfaces of the electronics interface portion 110 including the electrical pads 112 and electronic circuitry bonded thereto, can be coated with or encased or packaged in one or more types of biocompatible electrically insulating materials such as a non-conductive polymers (e.g., silicone) as needed for electrical isolation purposes, in a manner readily understood by individuals having ordinary skill in the relevant art.

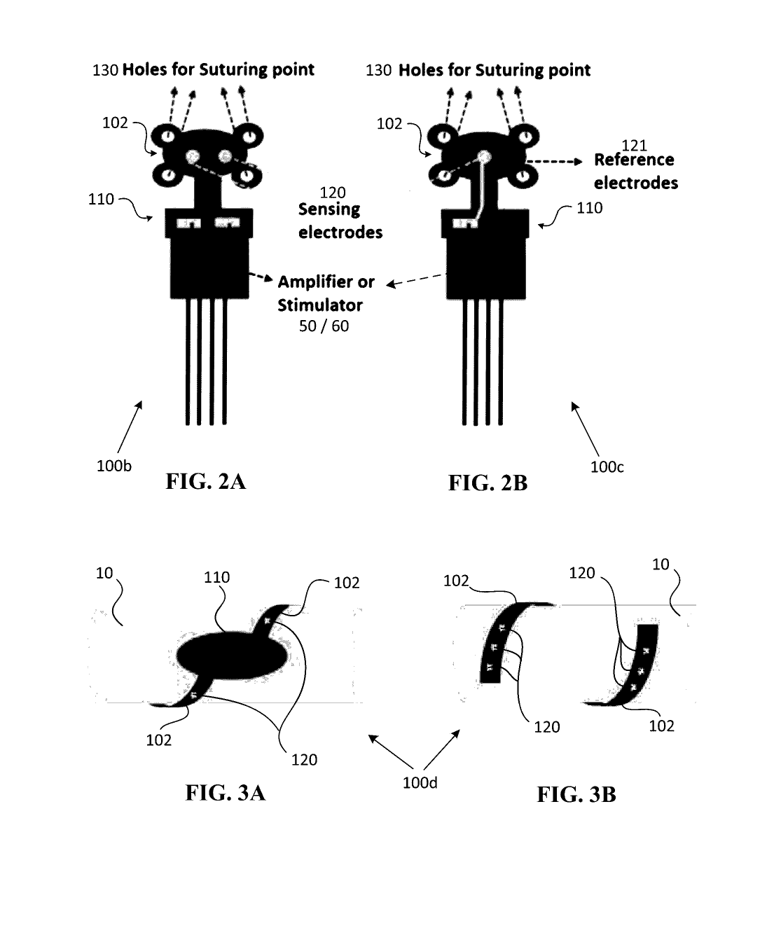

Flexible epineural strip electrodes 100 can exhibit a variety of other configurations, shapes, and sizes, as further elaborated upon hereafter with respect to FIGS. 2A-5C. FIGS. 2A and 2B illustrate representative flexible epineural strip electrodes 100b,c in accordance with alternate embodiments of the present disclosure, in which the nerve interface portion 102 is formed as an oval-shaped or generally oval-shaped member 104. In the embodiments shown, the oval-shaped member 104 carries two electrodes 120. For instance, such electrodes 120 can include a sensing electrode 120 and a reference electrode 121 as shown FIG. 2B. The electrodes 120, 121 reside on the inner surface of the flexible epineural strip electrode 100b,c, which contacts the epineurium when the flexible epineural strip electrode 100b,c is positioned or mounted on or secured to a peripheral nerve.

FIGS. 3A and 3B illustrate a representative flexible epineural strip electrode 100d in accordance with another embodiment of the present disclosure. In this embodiment, the nerve interface portion 102 includes two flexible spiral arm members 106 that spirally extend outward from the electronics interface portion 110, and which are configured to spirally wrap around an underlying peripheral nerve on which the electronics interface portion 110 is positioned.

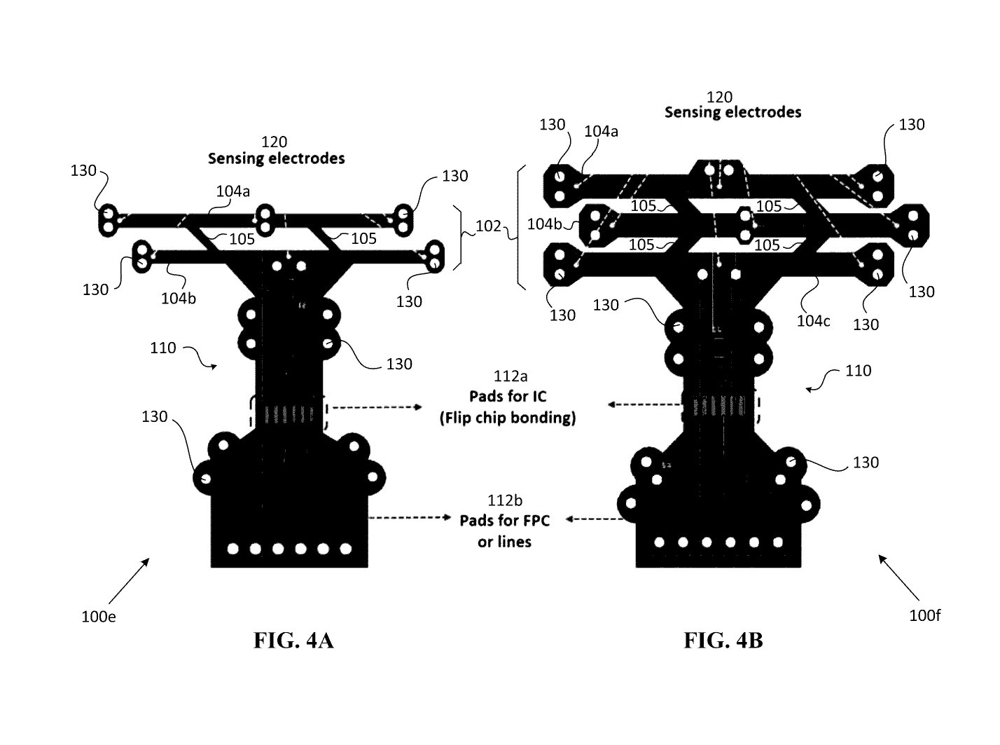

FIGS. 4A and 4B illustrate representative flexible epineural strip electrodes 100e,f in accordance with further embodiments of the present disclosure, in which the nerve interface portion 102 includes or provides multiple flexible elongate strips 104, each of which carries a set of electrodes 120. More particularly, the embodiment shown in FIG. 4A includes two flexible elongate strips 104a,b disposed parallel or generally parallel to each other, where each flexible elongate strip 104a,b carries three electrodes 120; and the embodiment shown in FIG. 4B includes three flexible elongate strips 104a-c disposed parallel or generally parallel to each other, where each flexible elongate strip 104a-c carries three electrodes 120. Individuals having ordinary skill in the relevant art will clearly understand that one or more flexible elongate strips 104 can carry additional or fewer electrodes 120 depending upon embodiment details. Each flexible elongate strip 104 can be structurally coupled to an adjacent flexible elongates strip 104 by a set of arm members 105, and each flexible elongate strip 104 any given arm member 105 can carry integrated circuit wiring by which one or more particular electrodes 120 are electrically coupled to the electrical interface portion 110. The presence of multiple flexible elongate strips 104 can enable better neuroelectric signal discrimination among fascicles when the flexible elongate strips 104 of the flexible epineural strip electrode 100e,f are positioned on a peripheral nerve. Depending upon embodiment details and neural amplifier or neural stimulator capabilities, the electrodes 120 of each flexible elongate strip 104 can be simultaneously active at one or more times; or the electrodes of any given flexible elongate strip 104 can be selectively active at one or more times relative to the electrodes 120 of an adjacent flexible elongate strip 104 (e.g., on a multiplexed basis), in a manner readily understood by individuals having ordinary skill in the relevant art. Each flexible elongate strip 104 includes a plurality of suture apertures 130 formed therein to facilitate suturing of the elongate strip 104 to a peripheral nerve, one or more other anatomical structures, and/or itself in a manner analogous to that described above. Additionally or alternatively, each flexible elongate strip 104 can be adhered to the peripheral nerve by way of a biocompatible adhesive and/or a bio-gel.

In the embodiments shown in FIGS. 4A and 4B, the electronics interface portion 110 includes multiple distinct sets of electrical pads 112, such as a first set of electrical pads 112a to which an integrated circuit can be bonded by way of a conventional flip-chip process; and a second set of electrical pads 112b to which an FPC can be bonded.

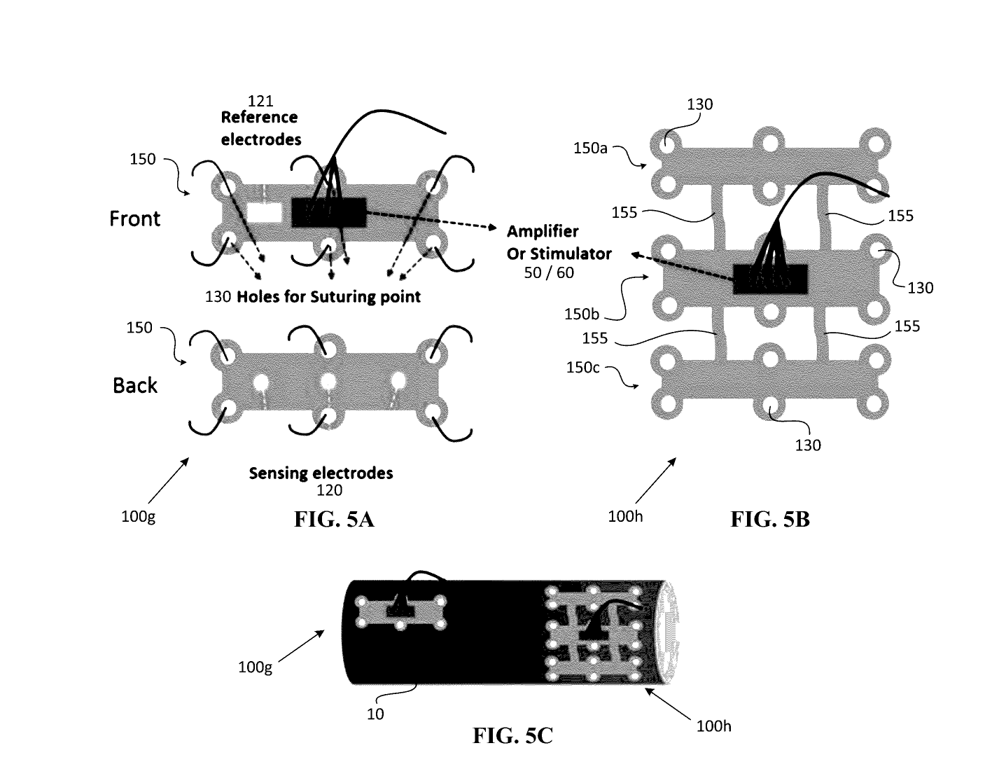

FIGS. 5A-5C illustrate representative flexible epineural strip electrodes 100g,h in accordance with additional embodiments of the present disclosure. The embodiment shown in FIG. 5A includes a single flexible strip 150 having a front, outer, or outward facing side or surface 152 configured for carrying a neural amplifier 50 or a neural stimulator 60, and at least one reference electrode 121; and a back, inner, or inward facing side or surface 154 that carries a set of sensing or stimulation electrodes 120. The flexible strip 150 includes a plurality of suture apertures 130 disposed about its periphery, such that the strip 150 can be sutured to an underlying peripheral nerve, another anatomical structure, and/or itself with its back or inner side 154 residing against the epineurium. The neural amplifier 50 or neural stimulator 60 can be electrically coupled to other circuitry in a conventional manner, for instance, by way of bond wires or lead wires.

The embodiment shown in FIG. 5B includes multiple spatially offset flexible strips 150a-c disposed in a parallel or generally parallel arrangement relative to each other, and which are structurally coupled to each other by arm members 155 that extend between the flexible strips 150a-c. Such spatially offset flexible strips 150a-c and arm members 155 form or generally form a type of grid or scaffold structure. A central flexible strip 150b can be configured to carry a neural amplifier 50 or a neural stimulator 60 on a front side thereof; and the central flexible strip 150b as well as outer flexible strips 150a,c carry electrodes (not shown) on their respective back sides. FIG. 5C illustrates representative manners in which the flexible epineural strip electrodes 100g,h of FIGS. 5A and 5B can be positioned upon a peripheral nerve, such that the electrodes on the back or inner sides of the flexible elongate strips 150a-c directly reside upon portions of the epineurium.

The flexible substrates of each of the representative flexible neural electrode embodiments 100a-100h shown in FIGS. 1-5C can be fabricated from conventional flexible biocompatible materials (e.g., polyimide, parylene, and/or another conventional biocompatible material) and the electrodes 120 can be fabricated from conventional biocompatible metals (e.g., gold, platinum, platinum-iridium, or platinum black) and/or biocompatible conductive polymers (e.g., poly(3,4-ethylenedioxythiophene) (PEDOT)), and possibly biocompatible nanomaterials/nanostructures coated thereon. Such fabrication can occur by way of conventional integrated circuit and/or microelectromechanical system (MEMS) fabrication techniques.

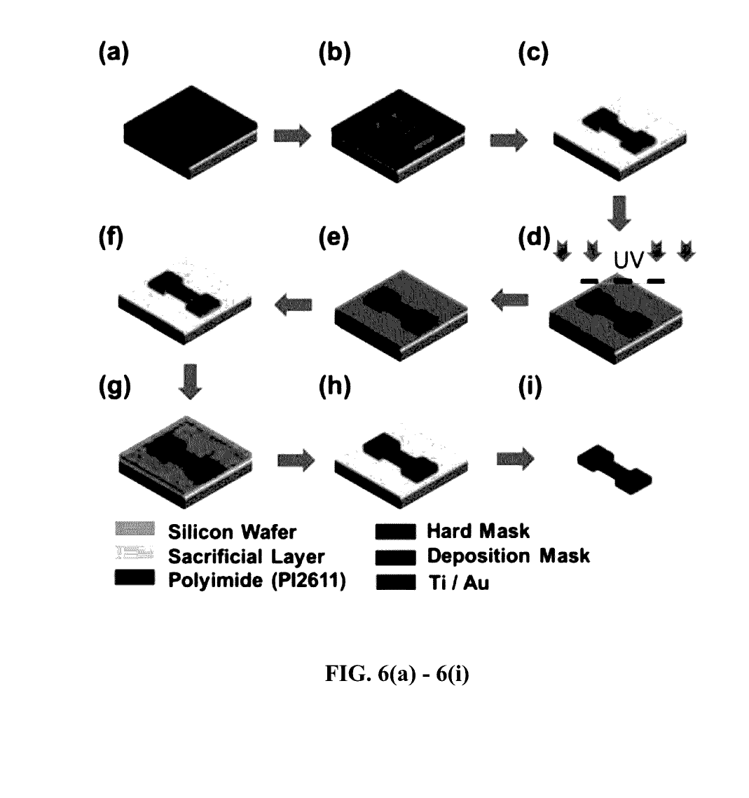

FIGS. 6(a)-6(i) illustrate aspects of a representative process by which a flexible epineural strip electrode 100 was fabricated in accordance with an embodiment of the present disclosure. The flexible epineural strip electrode 100 was made of two layers of polyimide, with gold sandwiched therebetween in a strip-shaped geometry. First, a 1 μm Aluminum (Al) sacrificial layer was deposited on a silicon substrate, then a 6 μm polyimide (PI2611-HD-Mircrosystems) was spin coated and hard cured under 300° C. for 30 minutes at a 4° C./min ramping rate (FIG. 6(a)). After deposition of 200 nm Al as a hard mask, the bottom polyimide structure was patterned on the thin Al surface after the first lithography step (FIG. 6(b)). Afterward, the exposed polyimide was etched out by a reactive plasma etching (ME) process (O₂ gas flow 50 sccm and CF₄ gas flow 10 sccm, RF power 150 W), the remaining Al layer was removed (FIG. 6(c)). Layers of Titanium (Ti) (20 nm) and gold (Au) (300 nm) metal electrodes, plus traces and pads were subsequently deposited and patterned by a metal evaporation and lift-off process (FIGS. 6(d)-(f). Next, a second layer of 6 μm polyimide was coated and fully cured at 350° C. for 30 min (FIG. 6(g)). Using the same process as the first polyimide layer patterning, the second polyimide layer was etched and the final structure was formed (FIGS. 6(h)-(i)).

As part of the fabrication of the representative flexible epineural strip electrode 100, surfaces of electrodes 120 were coated with Multi-walled Carbon Nanotubes (MWCNTs) to improve electrical performance. In particular, the MWCNTs (Cheap Tubes Inc., US, length ˜0.5-2 μm, outer diameter <8 nm) were first dispersed in an Au electrolyte bath (TSG-250, Transene, US) to form a 1 mg mL⁻¹ aqueous solution. Then, the whole solution was sonicated for 2 hours to fully suspend the CNTs in the solution. After that, the packaged flexible epineural strip electrode 100 and Au wire were connected to the negative and positive terminals of a power supply, respectively. Electrodes 120 and the Au wire were then inserted into the solution. A monophasic voltage pulse (1.1V, 50% duty cycle, 1 min) was applied from the power source. Au ions in the solution, as well as MWCNTs which absorbed Au ions, migrated to the negative terminals. After absorbing the electrons from the probe contacts, the Au ions were subsequently deposited onto the surfaces of the electrodes 120. The surface morphology of CNT coated electrodes 120 was characterized by Scanning Electron Microscopy (SEM).

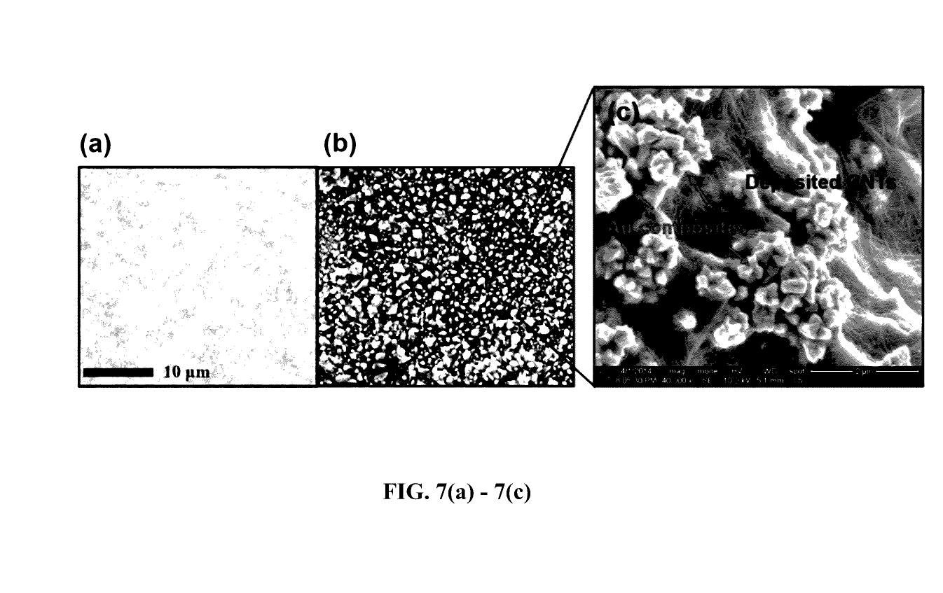

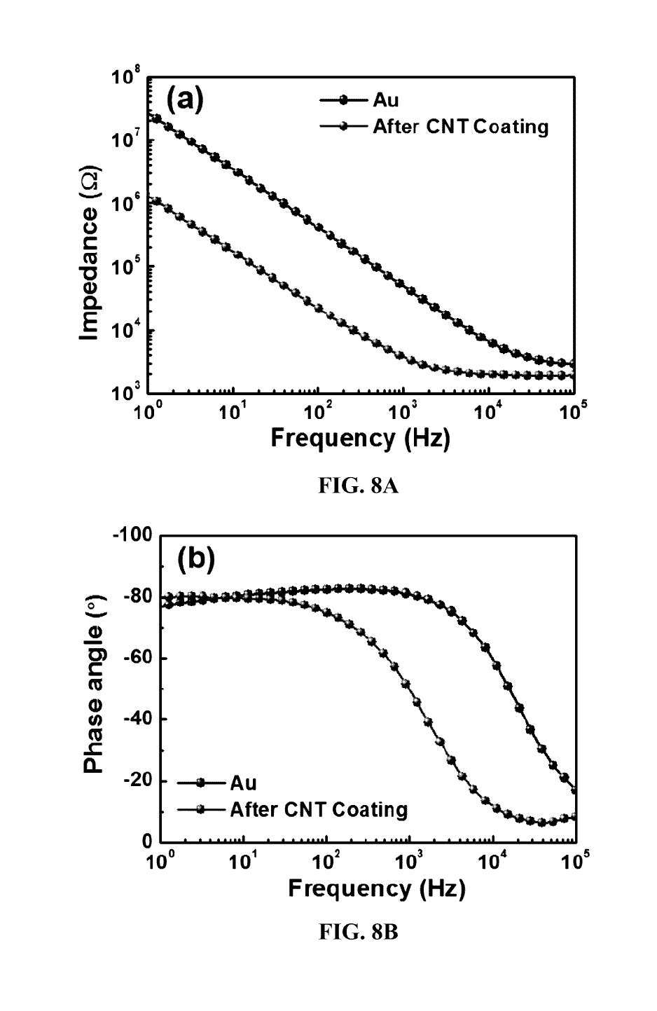

FIG. 7(a) is an SEM image showing an electrode surface prior to MWCNT coating; and FIGS. 7(b) and 7(c) are SEM images showing MWCNT coated electrode surfaces. Au nanocomposites mixed with CNTs were formed on the electrode surfaces, demonstrating that CNT-Au nanocomposites were successfully coated on the electrode surfaces by way of the CNT coating technique described above. The MWCNT surface coatings exhibited high roughness and high porosity, which provides increased electrochemical surface area (ESA) resulting in a dramatic decrease in the interfacial impedance of the electrodes 120. Impedance characterization of electrode interfaces or interfacial layers is important for the performance of neural recording, as the neural signal will be lost in noise if the electrode impedance is not sufficiently low. In order to verify the change of interfacial impedances with and without Au-CNT coated electrodes, electrochemical impedance spectroscopy (EIS) was conducted in phosphate buffered saline (PBS, Biowest, pH 7.4, conductivity×1). A sinusoidal wave with amplitude of 50 mV and frequency spans from 100 kHz to 0.7 Hz was applied. Three electrode configurations using a silver/silver chloride (Ag/AgCl) electrode and a Pt wire as reference and counter electrode, respectively, were used. The output impedance was recorded in vitro with an impedance analyzer (Autolab PGSTAT100N voltage potentiostat/galvanostat, Metrohm). The results of EIS are plotted in FIGS. 8A-8B. Impedance without CNT coating at 1 kHz was 47±7 kΩ, while the impedance dropped by approximately one order of magnitude (3.6±0.6 kΩ) after deposition of the CNT and Au composites; and phase was −48° at 1 kHz. Au electrodes with CNT coating have low to reasonably low interfacial impedance (<10 kΩ), and can be used to sense or record high quality neural signals.

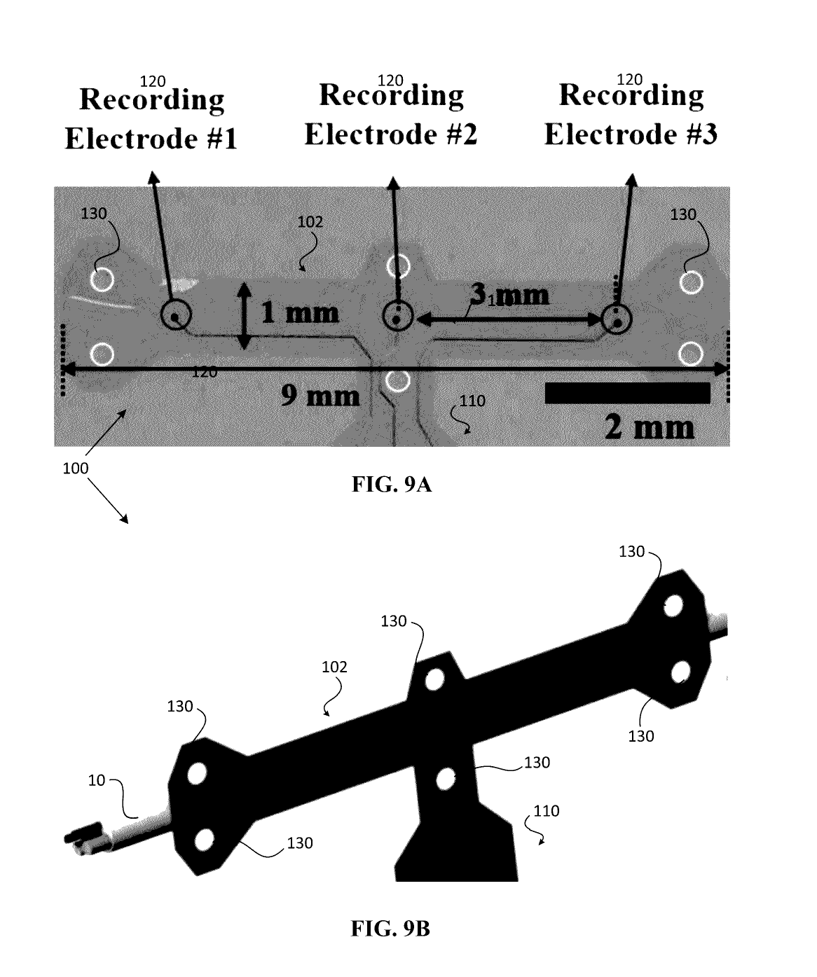

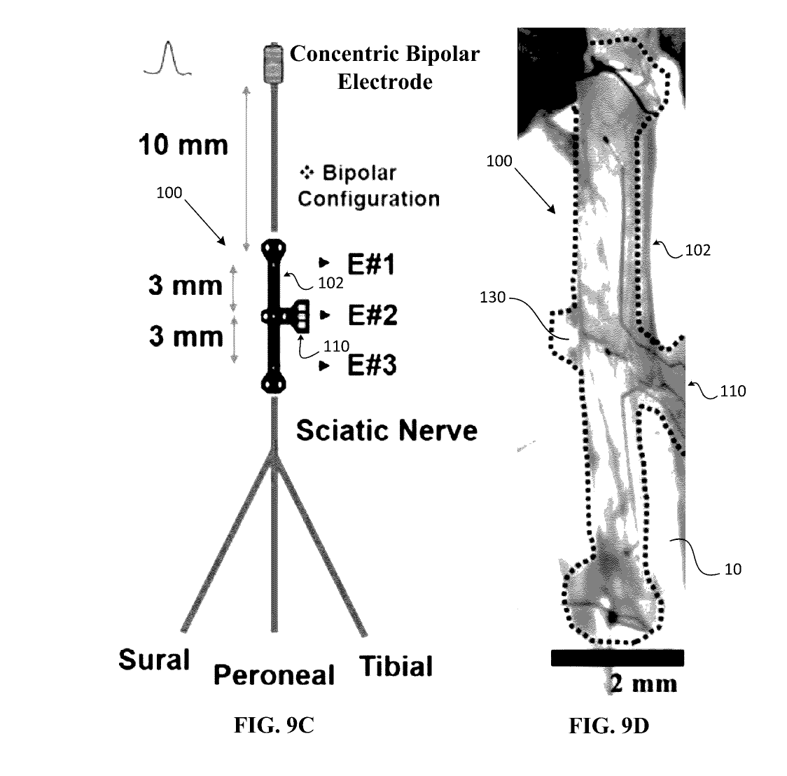

FIG. 9A is a photograph of an as-fabricated flexible epineural strip electrode 100, and dimensions thereof. This flexible epineural strip electrode 100 includes three electrodes 120, and six suture apertures 130 in a manner essentially identical or analogous to that described above with respect to FIG. 1. FIG. 9B schematically illustrates how this representative flexible epineural strip electrode 100 can be positioned upon a peripheral nerve prior to suturing the flexible epineural strip electrode 100 thereto. FIG. 9C schematically illustrates an experimental study setup in which this as-fabricated flexible epineural strip electrode 100 of FIG. 9A was positioned on a proximal segment of a rat sciatic nerve, where the three electrodes 120 of FIG. 9A are identified as E#1, E#2, and E#3; and FIG. 9D is a photograph showing this as-fabricated flexible epineural strip electrode 100 sutured to the rat sciatic nerve.

More particularly, an experiment was performed in adult female Sprague Dawley rats (250 g) (In Vivos Pte Ltd, Singapore). The rats were acclimatized for one week prior to use in the experiment, with food and water provided ad libitum and 12 h lights on/off. The animal care and use procedures conformed to those outlined by the Agri-Food & Veterinary Authority (AVA) of Singapore, the Institutional Animal Care and Use Committee (IACUC), and the ethics commission of the National University of Singapore. The animals were anesthetized with a single bolus injection of ketamine/xylazine (150 mg/kg and 10 mg/kg, respectively, intraperitoneal). After an adequate depth of anesthesia was attained, the right sciatic nerves were exposed through a gluteal-splitting incision. The flexible epineural strip electrode 100 was placed around the proximal segment of the sciatic nerve and sutured thereto by way of microsurgical techniques. Special care was taken to prevent nerve damage.

Neural signals were evoked by electrical stimulation during acute recording tests (studies done under general anaesthesia); evoked activity was used for testing the neural signal recording as well as the calculation of nerve conduction velocity (NCV). In this experiment, the rat sciatic nerve was directly stimulated and the evoked compound nerve action potentials (CNAPs) were recorded from the sciatic nerve by way of the three electrodes 120a-c of the flexible epineural strip electrode 100. The nerve was stimulated by the application of a single monophasic 20 μs pulse, with amplitudes varying between 0.3-1.5 mA, using an isolated stimulator box (Digitimer Ltd., UK). Signals from the implanted flexible neural electrode 100 were acquired using a multichannel amplifier (USB-ME32-FAI System, Multichannel Systems, Inc, USA), at a sampling rate of 50 kHz and a gain of 2000. Data acquisition was done using MCS system and data acquisition software (MCRack).

In this experiment, a concentric bipolar electrode (Microprobe, Inc) used as a stimulation electrode was implanted proximal to the spinal cord. Bipolar recordings were conducted using the three sensing electrodes 120a-c of the flexible epineural strip electrode 100 distally placed at about 10 mm distance from the stimulus site. A reference electrode was placed in the body in an electrically neutral place, and a ground electrode was separately connected to the tail of the rat.

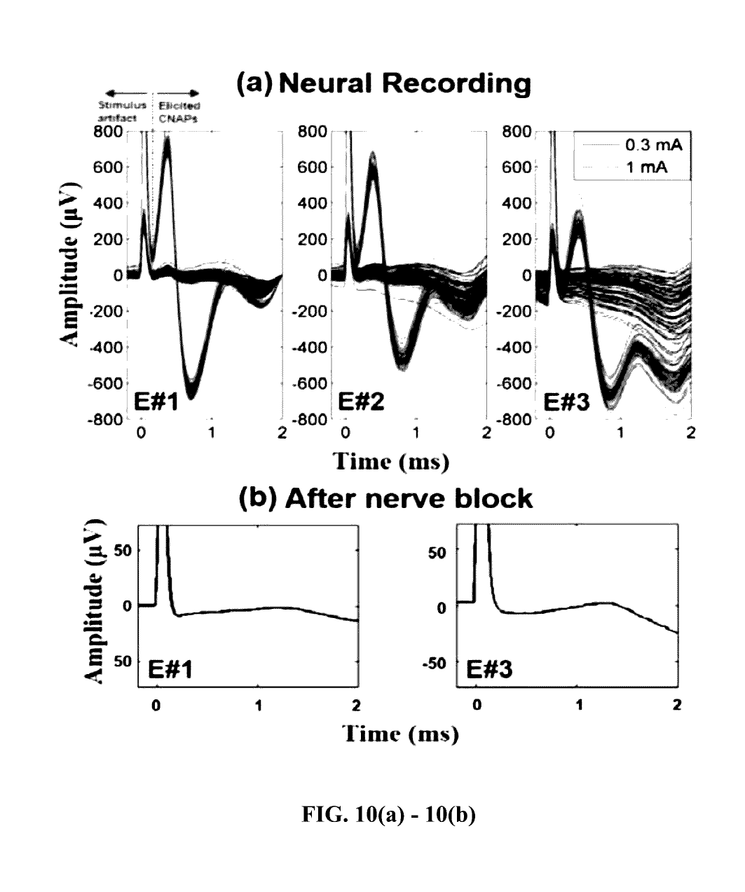

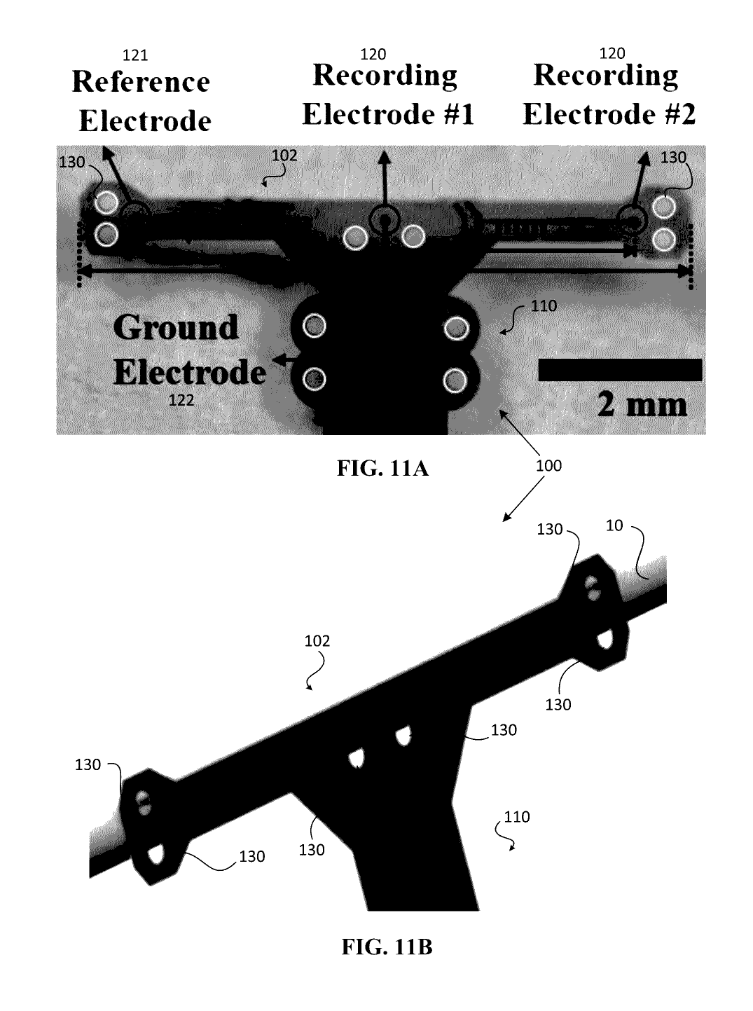

FIG. 10(a) shows recorded CNAPs from the three electrodes E#1, E#2, and E#3 for varying stimulation currents. Each trace corresponds to the average obtained from recording over 50 CNAPs. The CNAPs were recorded following the stimulation artifact: there were no CNAPs of significant amplitude recorded from the electrodes during the stimulation of 0.3 mA, whereas clear and fine neural signals were recorded from the three electrodes at 1 mA, and after the stimulation currents of more than 1 mA, the amplitude of recorded signals did not increase. This experiment demonstrates that stimulation current of around 1 mA is the threshold for stimulation of the sciatic nerve by the concentric bipolar electrode. The latency of peak of the CNAP that indicates the time from the onset of the stimulation artifact to the onset of the CNAP were 0.41, 0.42 and 0.44 ms, respectively, from the three electrodes. Nerve conduction velocity (NCV) was calculated in a conventional manner to be approximately 24-36 m/s. The mean amplitude of CNAP from E#1 was 716.15±39.74 μV, that of E#2 was 584.28±42.9 μV and that of E#3 was 272.94±50.55 μV. This can be attributed to the difference in the distance between the sensing electrodes and the reference electrode.

To verify whether the recorded CNAPs are corrupted by external noises such as EMG or the source of stimulation, xylocaine that is normally used for blocking nerve function was applied to the nerve during stimulation with 1 mA current and CNAP recordings were conducted after 10 minutes. The result of recorded CNAP after 10 minutes shows that no signal was recorded except for the stimulus artifact (FIG. 10(b)). This demonstrates that the recorded CNAP before nerve blocking are not corrupted. However, in this experiment the needle-type concentric electrode showed difficulty in making reliable and repeatable penetrations since some movements of nearby muscles during the stimulation made the concentric electrode lose its original position. Also, repeated penetration at the same stimulus position caused some sciatic nerve bleeding, degrading the normal nerve condition.

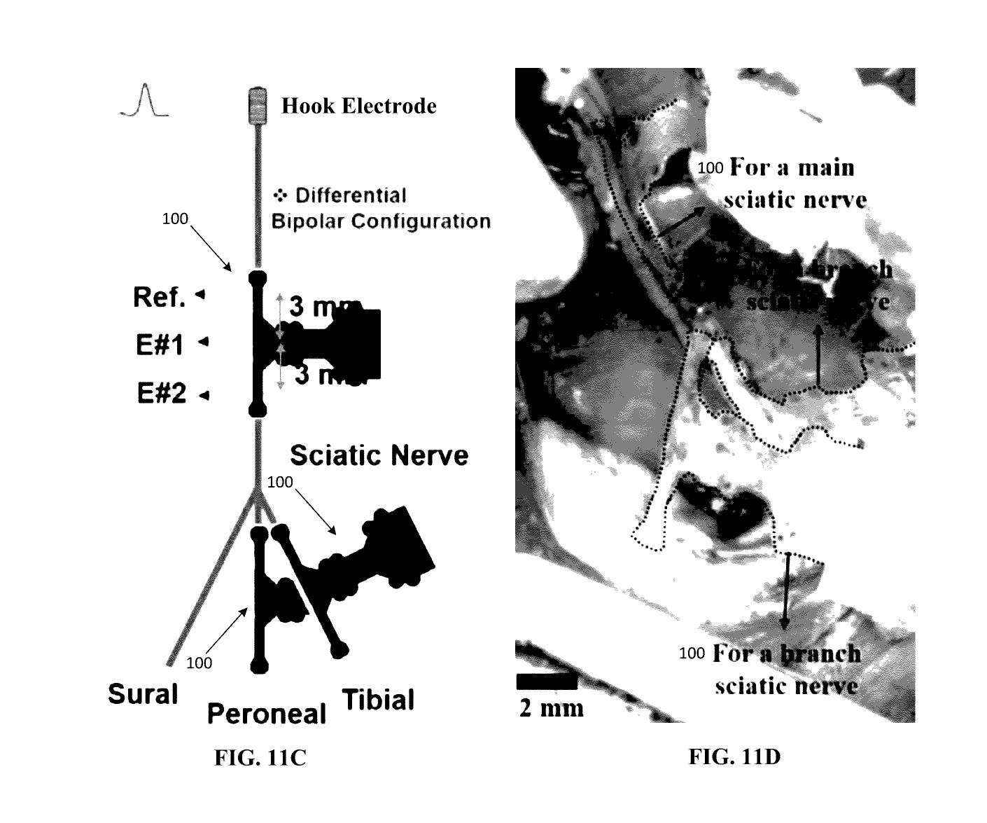

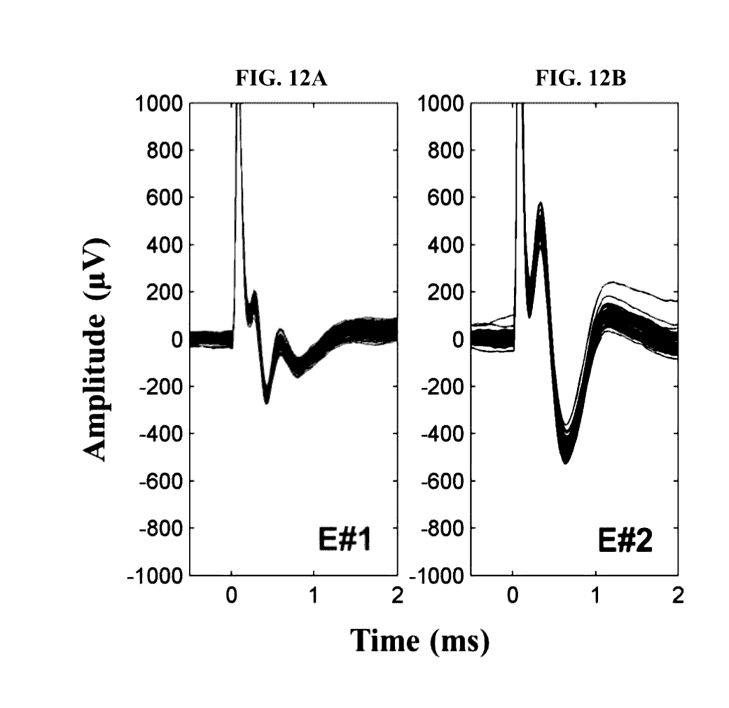

FIG. 11A shows another as-fabricated flexible epineural strip electrode 100 and its dimensions. This flexible epineural strip electrode 100 includes a reference electrode 121 plus two sensing electrodes 120 carried by its nerve interface portion 102; and a ground electrode 122 carried by its electronics interface portion 110. The as-fabricated flexible epineural strip electrode 100 of FIG. 11A has a narrower nerve interface portion 102 than that of the as-fabricated flexible strip electrode 100 of FIG. 9A. As a result, the as-fabricated flexible epineural strip electrode 100 of FIG. 11A can easily stick even on small peripheral nerve branches, such as smaller branches of sciatic nerves (e.g., having diameters of 0.2˜0.3 mm). FIG. 11B schematically illustrates a representative manner in which the as-fabricated flexible epineural strip electrode 100 of FIG. 11A can be positioned on a peripheral nerve.

FIG. 11C schematically illustrates an experimental study setup in which one as-fabricated flexible epineural strip electrode 100 of FIG. 11A was positioned on a proximal segment of a rat sciatic nerve, and two additional as-fabricated flexible epineural strip electrodes 100 of FIG. 11A were positioned on peroneal and tibial branches of the rat sciatic nerve. In FIG. 11C, the reference electrode 121 of the as-fabricated flexible epineural strip electrode 100 of FIG. 11A is identified as Ref., and the two sensing electrodes 120 are identified as E#1 and E#2. FIG. 11D is a photograph showing the three as-fabricated flexible epineural strip electrodes 100 positioned on the proximal segment of the rat sciatic nerve, the peroneal branch of the rat sciatic nerve, and the tibial branch of the rat sciatic nerve. These as-fabricated flexible epineural strip electrode 100 exhibited good inherent adhesion to the epineurium on which they were positioned, and thus suturing was not actually required for retaining the as-fabricated epineural strip electrodes 100 in position during an acute neural signal recording experiment.

In the experiment corresponding to FIGS. 11C and 11D, a differential bipolar configuration was used for CNAP recording, which is a more effective recording technique in that electrical activity that is distant from the two sensing electrodes E#1 and E#2 appears as common mode, and is rejected; while electrical activity in the immediate vicinity of the two sensing electrodes E#1 and E#2 is differential mode and is amplified. Also, in this experiment a hook electrode was used as the stimulation electrode for more reliable and repeatable stimulation. The stimulation protocol was as described above. Threshold stimulation current was found to be around 1 mA, and clear and fine CNAPs were recorded. FIGS. 12A and 12B respectively show the result of recorded CNAPs (n=60) from the two sensing electrodes E#1 and E#2 positioned on the rat main sciatic nerve, elicited by the hook electrode with a stimulus current of 1 mA. The mean amplitude of CNAP from E#1 was 235.7±20.1 μV and that from E#2 was 466.1±34.6 μV. The difference in amplitude was most likely due to variability in the stimulation, slight slights in the nerve position, or placement of the as-fabricated flexible epineural strip electrode 100. Short distances between the reference electrode Ref and the sensing electrodes E#1 and E#2 compensates CNAP, resulting in low amplitudes. In the main sciatic nerve, a distance or gap of approximately 3 mm between the recording electrode #1 and #2 resulted in a 230 μV difference. For noise analysis, the mean amplitude of noise from the electrodes was 12.60±0.84 μV, which were identified before and after the CNAP.

### Flexible Neural Ribbon Electrode Structures

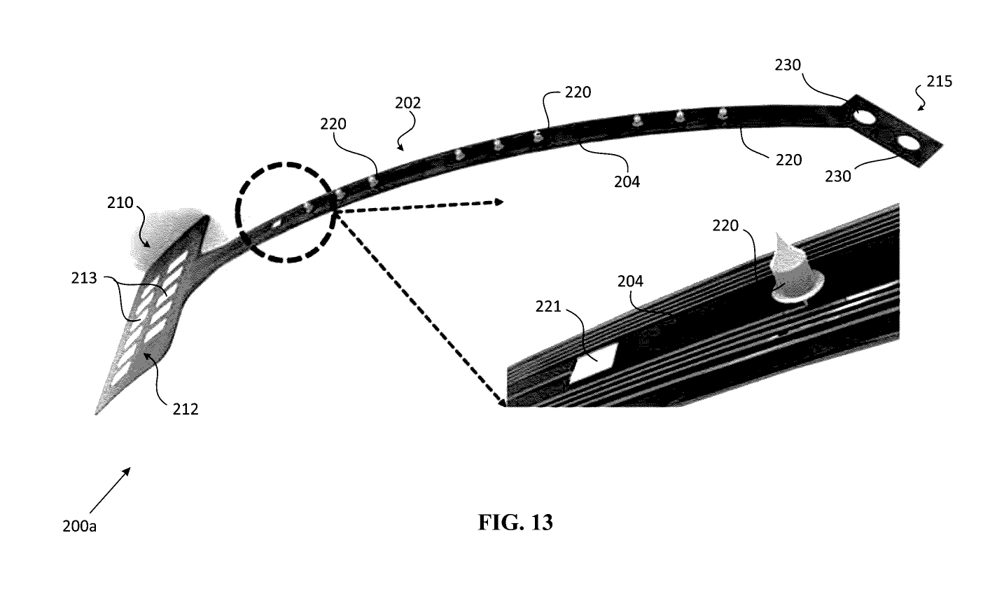

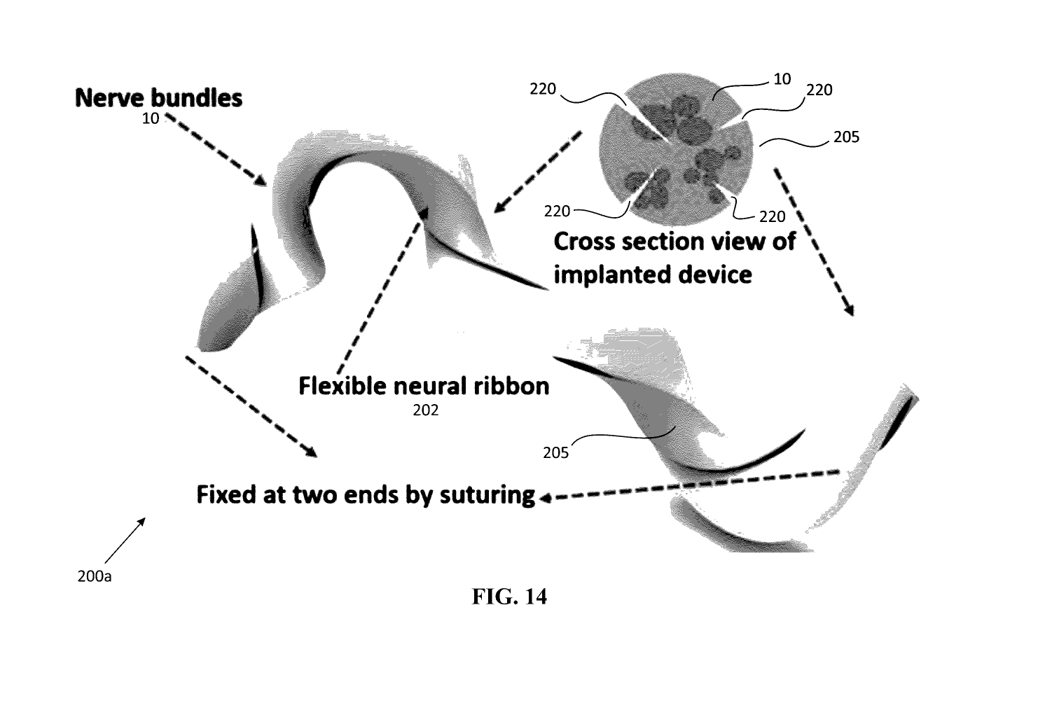

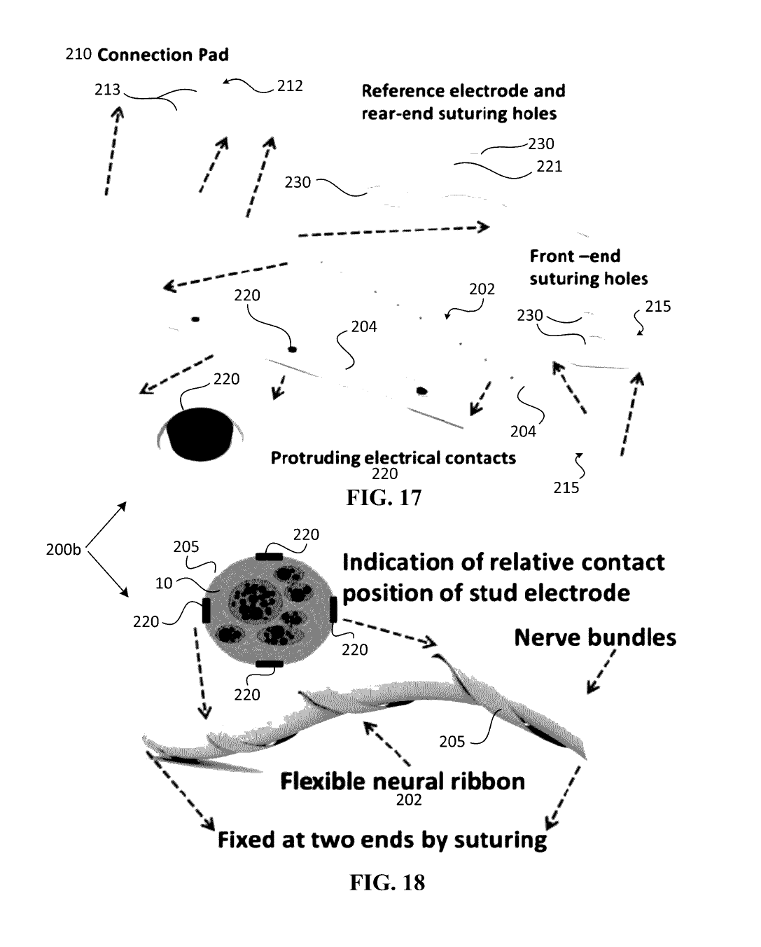

FIGS. 13 and 17 are schematic illustrations of representative flexible neural ribbon electrodes or electrode structures 200a,b in accordance with embodiments of the present disclosure. FIGS. 14 and 18 are schematic illustrations showing portions of the representative flexible neural ribbon electrodes 200a,b of FIGS. 13 and 17, respectively, positioned on a peripheral nerve 10. In an embodiment, the flexible neural ribbon electrode 200a,b includes a single, unitary, or unified flexible substrate providing a thin or very thin (for instance, less than approximately 30 microns thick, e.g., 10-20 microns thick) flexible elongate ribbon or flexible elongate ribbon section 202 that carries a plurality of sensing and/or stimulation electrodes 220 along its length, and along which integrated circuit wiring runs or extends; a back end portion or section 210 that provides a connection pad area, structure, or array 212 that includes a plurality of electrical pads 213 to which an electronic device distinct from the flexible neural ribbon electrode 200a,b can be electrically coupled or bonded; and a front end portion or section 215. The back end portion 210 and/or the front end portion 215 can include a plurality of suture apertures 230 formed therein, by which the back and/or front end portions 210, 215 can respectively be sutured to a peripheral nerve or peripheral nerve bundle. A reference electrode 221 can be carried by the back end portion 210 or a segment of the flexible elongate ribbon 202, such as a section of the flexible elongate ribbon 202 proximate to the back end portion 210.

The flexible neural ribbon electrode 202a,b includes an inner surface 204 that faces the peripheral nerve, and an outer surface 205 opposite to its inner surface 204 that faces away from the peripheral nerve. Correspondingly, the elongate ribbon 202 includes an inner surface 204 from which the sensing/stimulation electrodes 220 protrude; and an outer surface 205 opposite to its inner surface 204. The reference electrode 221 can be carried on the inner surface 204 of the flexible elongate ribbon 202, or on an inner surface of a section of the flexible neural ribbon electrode 202a,b near the back end portion 210.

The flexible elongate ribbon 202 is flexibly or resiliently coilable, windable, or wrappable along a spiral or helical path about (i) a longitudinal axis that runs parallel to or extends along the flexible elongate ribbon 202 between the back end portion 210 and the front end portion 215, (ii) a peripheral nerve or peripheral nerve bundle, or (iii) another anatomical structure. Thus, as shown in FIGS. 14 and 18, the flexible elongate ribbon 202 can be twined or spirally wound around portions of the epineurium along the length of a peripheral nerve or peripheral nerve bundle, such that the inner surface 204 of the flexible elongate ribbon 202 faces and contacts the peripheral nerve or peripheral nerve bundle, the electrodes 220 are arranged in a helical pattern along the peripheral nerve or peripheral nerve bundle, and the electrodes 220 directly electrically interface with peripheral nerve tissue underlying the flexible elongate ribbon 202. As a result, the flexible elongate ribbon 220 can automatically conform or self-adapt to peripheral nerves or peripheral nerve bundles of various diameters, including peripheral nerves or peripheral nerve bundles having small or very small diameters (e.g., 50-250 microns).

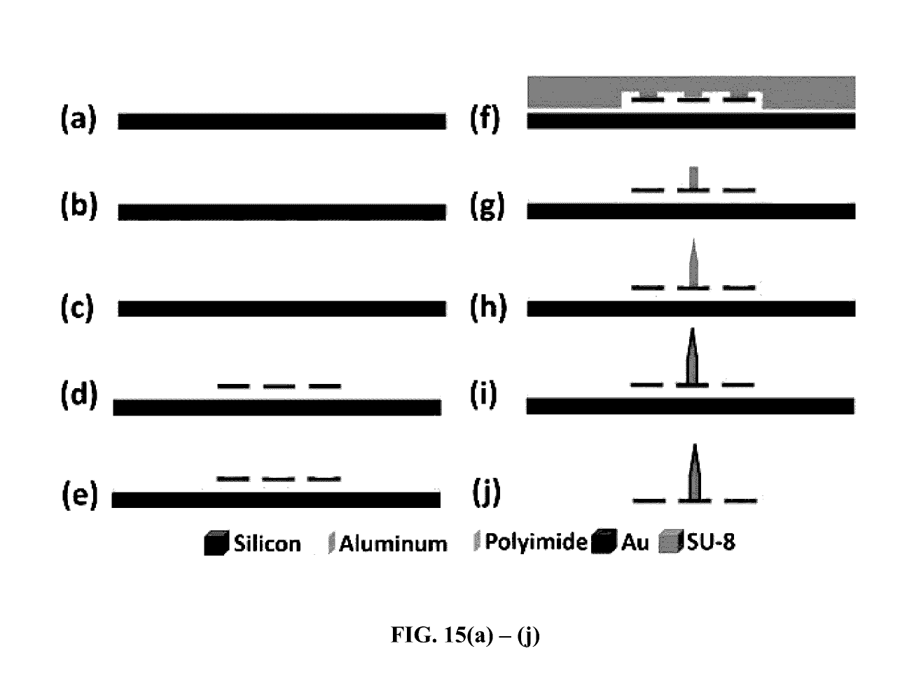

In some embodiments such as that shown in FIGS. 13-16B, the electrodes 220 include or are microneedle structures, which are configured to penetrate into peripheral nerve tissue, and which can aid anchoring of the flexible elongate ribbon 202 to the peripheral nerve or peripheral nerve bundle, and improve neural signal recording specificity and/or stimulation signal delivery specificity. Additionally or alternatively, in other embodiments such as that shown in FIGS. 17 and 18, the electrodes 220 include or are stud type structures that improve contact with the peripheral nerve without penetration and the associated risk of nerve damage. In various embodiments, electrodes 220 can include one or more types of coatings, such as nanomaterial or nanostructure coatings. FIGS. 15(a)-(j) illustrate aspects of a representative process by which a flexible neural ribbon electrode 200a that carries microneedle electrodes 220 can be fabricated in accordance with an embodiment of the present disclosure. In an embodiment, the flexible neural ribbon electrode 200a includes four layers, namely, a polyimide substrate layer; a conductive metal layer; a polyimide insulation layer; and SU-8 microneedle electrodes 220. After release from a Si substrate, the whole device becomes flexible because it is made of thin or very thin flexible polyimide. The SU-8 microneedle electrodes 220 have a length from 500 um up to 1 mm, and can be used to penetrate the peripheral nerve. The outer surfaces of the microneedle electrodes 220 include a biocompatible conductive layer (e.g., Au or Pt) coated thereon to aid signal acquisition. When the flexible neural ribbon electrode 200a is applied to a peripheral nerve or peripheral nerve bundle, neural signals from axons that are close to the microneedle electrodes 220 can be sensed.

Aspects of the fabrication process corresponding to FIGS. 15(a)-(j) are as follows:
(a) A Si wafer was cleaned by Acetone, IPA and DI water. Then it was dehydrated at a temperature of 180 degrees Celsius for 30 minutes. Next, a 1 μm Aluminum sacrificial layer was deposited on the Si substrate.
(b) The wafer was spin coated with a layer of Polyimide at 2000 rpm for 30 seconds. It was baked under 110 degree on the hotplate for soft baking.
(c) After UV lithography and development, the polyimide substrate can be defined as the device shape.
(d) With a standard liftoff process, the metal tracing, electrode contacts, and electrode pads were defined on the top of polyimide substrate.
(e) An insulation polyimide layer was defined on top of the metal layer.
(f) A 300 μm thick SU-8 layer was spun on the top of the polyimide layer. It was baked on a hot plate at 65 degree for 15 minutes, followed by 95 degree for 4 hours in a soft baking process.
(g) After the lithography process and development, SU-8 pillars were defined on the top of electrode contacts.
(h) By drawing lithography technology, the sharp tips of microneedles can be integrated on the top of these micropillars.
(i) With the help of a shadow mask, a layer of 50 nm Ti and 250 nm Au can be sputtered on the top of SU-8 microneedles for conductive sensing electrodes.
(j) By using anodic metal dissolution, the sacrificial Aluminum layer can be dissolved in the solution, and the whole device can be released from the underlying Si wafer substrate. Then the device was integrated with an FPC connector for testing purposes.

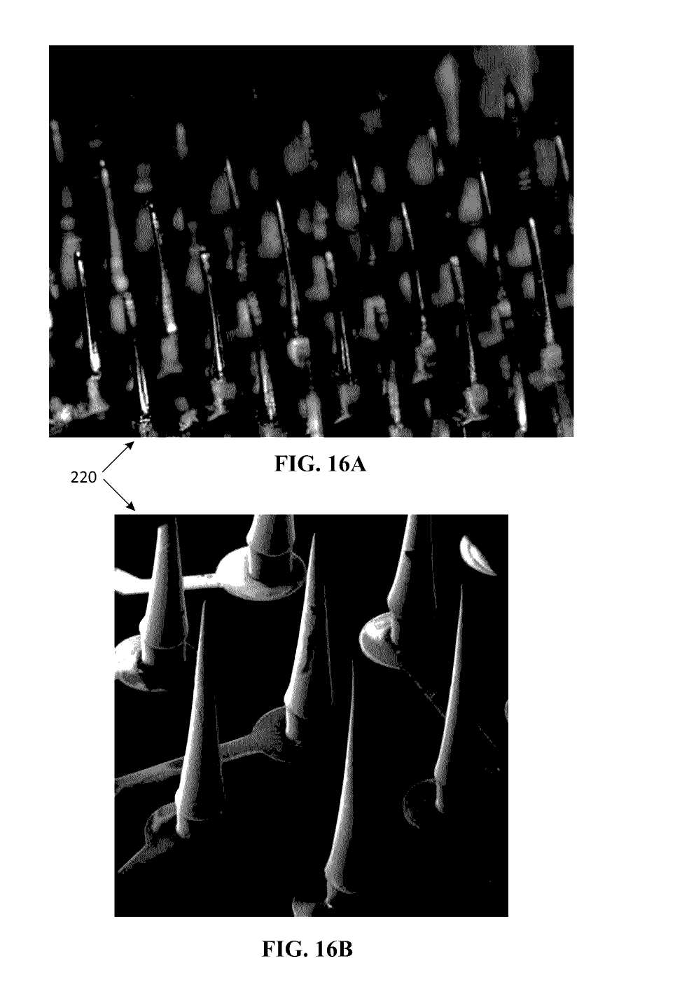

FIGS. 16A and 16B show an optical image and a SEM image, respectively, of the as-fabricated microneedle electrodes 220 of FIGS. 14 and 15.

FIGS. 17 and 18 illustrate another embodiment of a flexible neural ribbon electrode 200b, which includes 3D stud type electrodes 220 that avoid penetrating into the epineurium. The studs bridge the small gap between the metal surface and the nerve introduced by the polyimide insulation layer. In addition, the studs can allow the neural ribbon electrode to maintain electrical contact with the peripheral nerve even in cases of a slight delamination of the electrode from the nerve. The flexible neural ribbon electrode 200b including its flexible elongate ribbon 202 can be fabricated from an ultra-thin polyimide substrate, such as Durimide. Wrapping the flexible neural ribbon electrode 200b around a peripheral nerve or peripheral nerve bundle will not compromise the structural integrity of the Durimide substrate.

In a representative as-fabricated implementation, two front-end suturing holes 230 can be used to fix the front part 215 of the flexible neural ribbon electrode 200b on the surface of the epineurium. Eight 3D circular protruding electrodes 220 having a diameter of 150 μm reside on a 1.4 cm long flexible elongate ribbon or stripe 202, which serves as the main body of the device to communicate with nerve bundles. A 200 μm×500 μm reference electrode 221 and four rear-end suture holes 230 lie on two small wing portions. The four suture holes 230 are designed to fix the rear part of the device on the epineurium. In order to minimize interference from a connector during implantation, a 0.5 cm transition region is intentionally added between the connection pad 210 and the rear-end suture holes 230. At the other terminal of the flexible neural ribbon electrode 200, a special connection pad with through holes is designed to match with a customized connector. In the practical implantation procedure, the device 200b is designed to be attached on the nerve as shown in FIG. 18. The front-end suture holes 230 are used to fix the front part of device 200b on the peripheral nerve. With this fixed part, the flexible elongate ribbon 202 can be helically wrapped along the peripheral nerve due to the high flexibility of the ultra-thin polyimide substrate. The 3D circular protruding electrodes 220 directly touch the epineurium surface, which establishes an excellent communication between sensing contacts and activated nerve bundles.

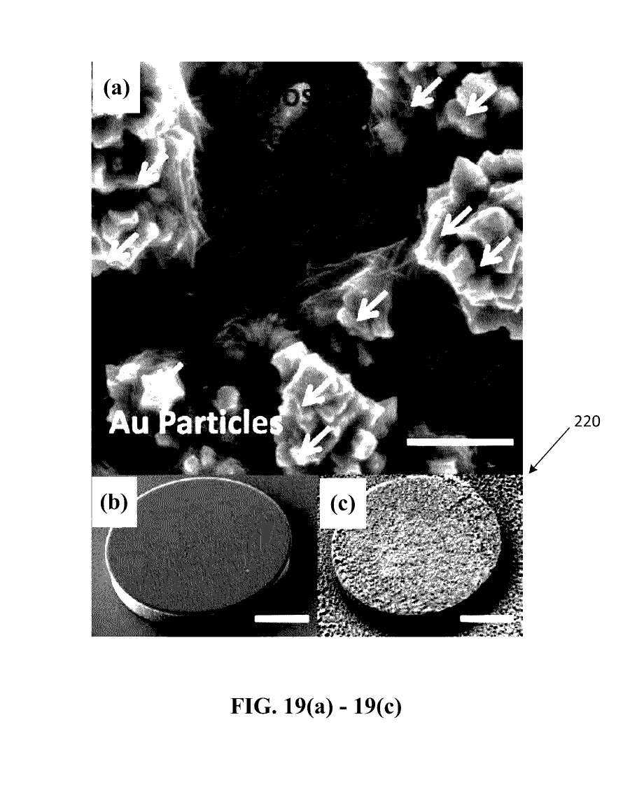

The 3D stud-type electrodes 220 are fabricated from SU-8, onto which a layer of CNTs is coated to increase the effective surface area and improve charge transfer at the electrode-tissue interface. An electrophoretic deposition (EPD) technique was employed to deposit the CNT film since it is an automated high-throughput process that in general produces films with good homogeneity and packing density. Under an applied electrical voltage, Au ions in the solution as well as CNTs that absorbed Au ions migrate to the negative terminals. After getting the electrons from the protruding contacts, Au ions are subsequently deposited on the contact surface. The CNTs with a diameter of 0.5-2 μm and a length less than 8 μm also adhere on the Au electrode contacts by these ions. FIG. 19(a) is an SEM image of deposited Au particles and CNTs. An Au coated electrode 220 and a CNT coated electrode 220 are shown in FIGS. 19(b) and 19(c), respectively. Impedance spectroscopy of Au electrodes 220 and CNT coated electrodes 220 showed that at the biologically relevant frequency of 1 kHz, the impedance of the Au electrode 220 and the CNT coated electrode 220 were 285.47 kΩ and 6.2 kΩ, respectively. The neural ribbon electrode also showed a reversible linear elongation when applied strain was less than 7%. Due to the ultrathin polyimide layers, the bending stiffness of the fabricated device was less than 200 N/μm², which was conducive to the wrapping process in an in vivo experiment.

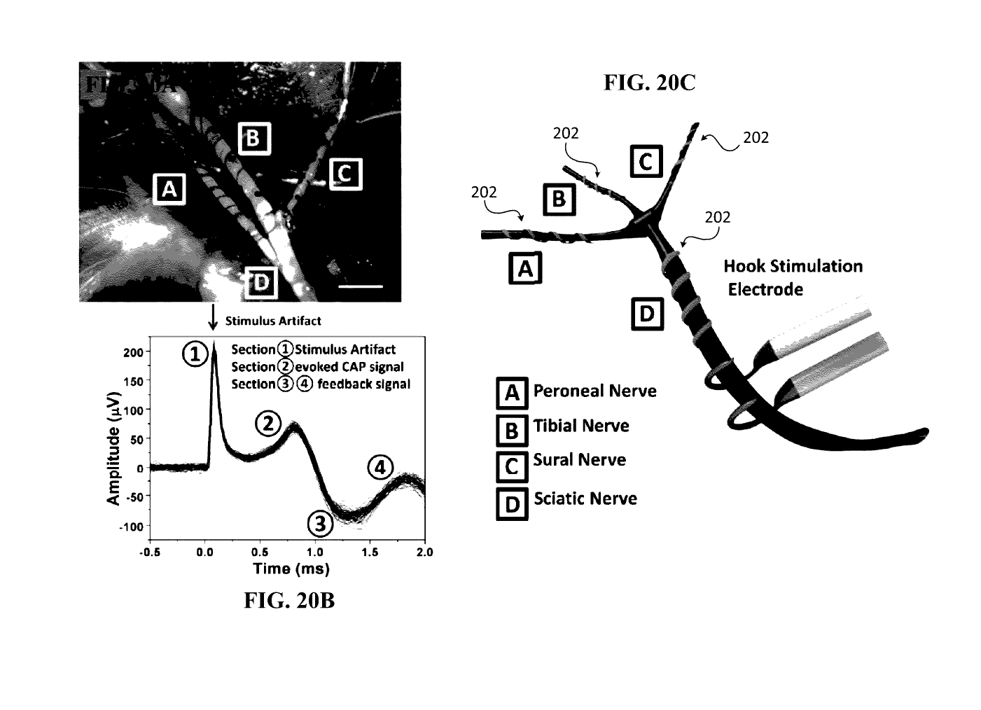

In order to demonstrate that the flexible neural ribbon electrode 200b is capable of adaptively matching with nerves of different diameters including small nerves, three terminal branches of a rat sciatic nerve in different diameters (300 μm˜600 μm), namely, the peroneal nerve, tibial nerve, and sural nerve, were implanted with flexible neural ribbon electrodes 200b as shown in FIGS. 20A and 20C. For assessment of nerve recording capabilities, acute recording experiments were conducted using the fabricated devices implanted on the foregoing portions of a rat sciatic nerve. The experimental set-up for evoking and recording compound action potentials (CAPs) on the nerve is shown in FIGS. 20B and 20C. CAPs were evoked by delivering 20 μs cathodic monophasic pulses of varying current (0.2 mA˜0.7 mA) through two hook platinum electrodes using a Digitimer stimulator. The responses evoked with the varying stimulus parameters were recorded by flexible neural ribbon electrodes 200b disposed distal to the hook electrodes.

More particularly, to demonstrate the recording capability of as-fabricated flexible neural ribbon electrodes 200b on small nerves with different diameters, four neural ribbon devices were implanted on the surface of the rat sciatic nerve, the peroneal nerve, tibial nerve and sural nerve. Differential recordings of the CAPs were taken from contacts on each flexible neural ribbon electrode 200b with respect to a ground Ag/AgCl wire sutured under the skin beside the surgical site. Sixty evoked CAPs every second were recorded and averaged to reduce noise. Complex waveforms were observed in these stimulated CAPs. FIG. 20B shows a representative signal recorded from one of the electrode channels. The recorded signal was divided into four parts. The simulation was delivered at time 0 and the corresponding stimulus artifact appeared immediately. The stimulus artifact varied in duration and amplitude based on the stimulus intensity and pulse width applied through the hook stimulation electrode. The peak that followed was defined as the directly evoked CAP signal conducted by the nerve that was marked as Section 2 in FIG. 20B. Section 3 and Section 4 in FIG. 20B were possibly electrical signals from nearby muscles or compound sensory signals (nerve fascicles are known to contain both motor and sensory axons) triggered by the stimulation.

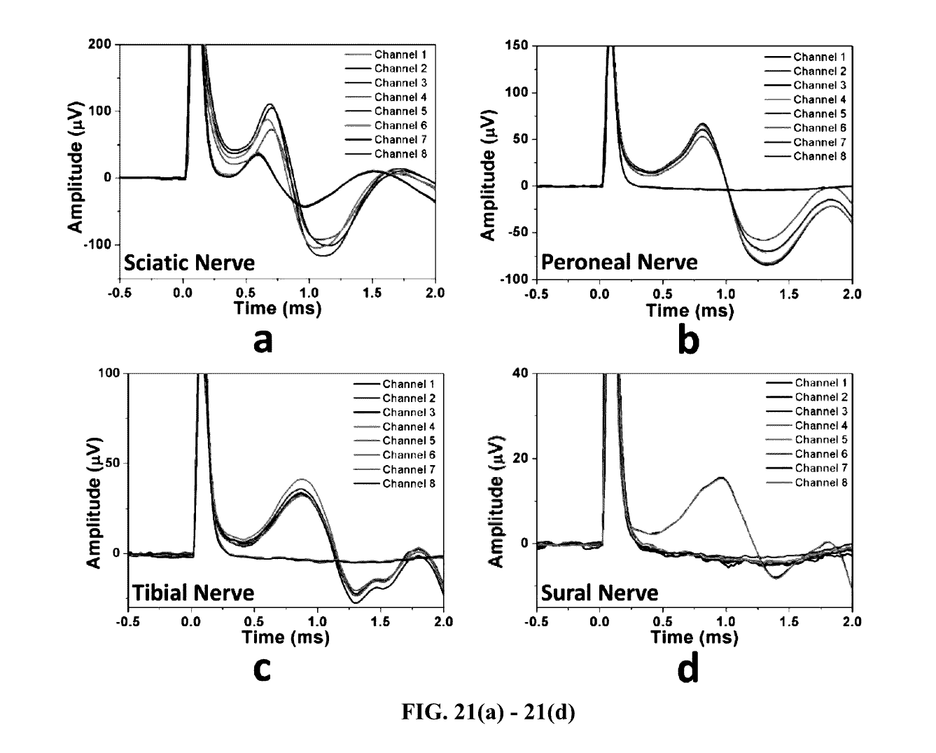

The recorded signals under 0.6 mA stimulation from 4 different neural ribbon electrodes are shown in FIGS. 21(a)-(d). FIG. 21(a) was the signal recorded from the sciatic nerve. All the 8 electrodes 220 on the neural ribbon were activated but the amplitudes were different. Since the fascicles had an anisotropic distribution under epineurium, the distance between sensing electrodes 220 on the neural ribbon and active fascicles varied at different locations. Thus, even under the same stimulation, the conductive current density received by the electrodes 220 was different. Meanwhile, not all the electrodes 220 of the neural ribbon electrodes that were implanted on the three branch terminals could be activated. FIG. 21 (b)-(c) show that only some of the channels could record neural activities. Especially on the smallest sural nerve, only two channels were activated. Since the nerve fascicles in sciatic nerve split into three portions, the number of fascicles inside branch terminals was smaller than that in sciatic nerve. When the nerve was stimulated under the same current, the number of activated fascicles was the lowest in the smallest sural nerve. Only the electrodes 220 that were close enough to those activated fascicles in the sural nerve might receive sufficient current to record any signals. That is why only two channels recorded signals in the flexible neural ribbon electrode 220b that was attached to the sural nerve. This result also indicated that multiple electrodes 220 on a nerve can increase the probability of obtaining high-quality neural signals and allow for discrimination of individual neural signals, leading to higher quality and higher fidelity neural signal discrimination and neural signal function decoding capabilities.

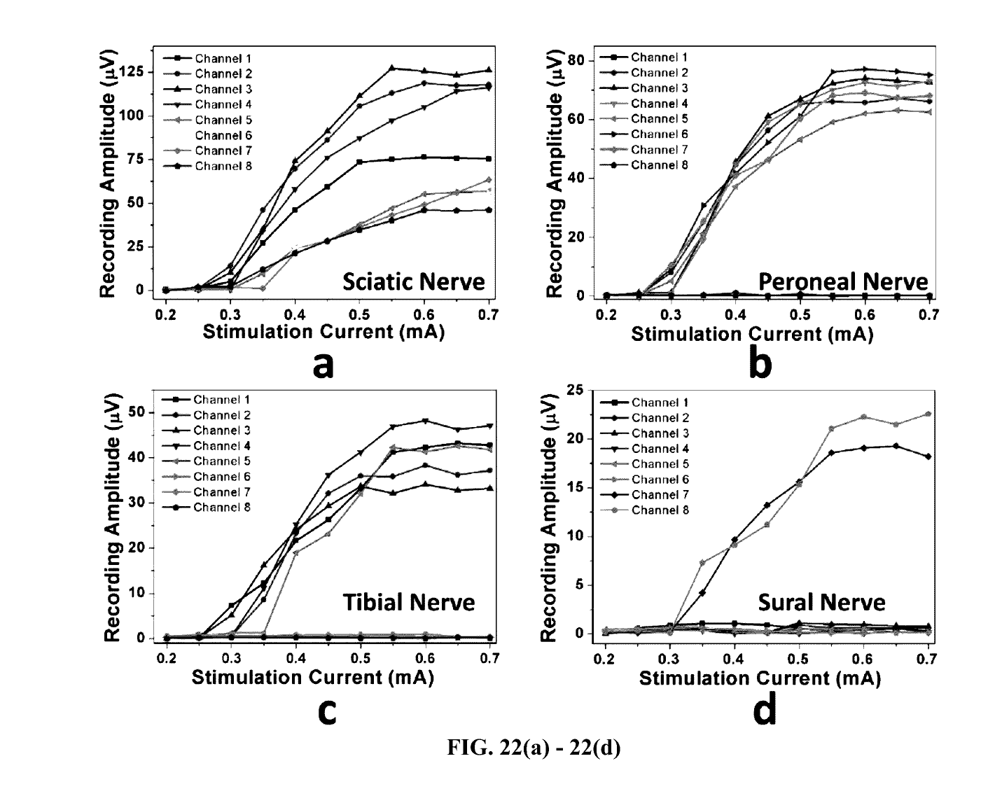

The peak value of neural activity was recorded with increasing stimulus intensity (from 0.2 mA to 0.7 mA). The results are shown in FIGS. 22(a)-(d). FIG. 22(a) shows the recording amplitude from the sciatic nerve, while FIGS. 22(b)-(d) show recording amplitudes from the peroneal nerve, tibial nerve, and sural nerve, respectively. The evoked CAPs were the algebraic summation of all the action potentials produced by all the fascicles within the nerve bundles excited by the electrical stimulation. When the stimulation current was lower than 0.3 mA, few fascicles were activated and most of the electrodes 220 on the flexible neural ribbon electrodes 200b could not record any signals. When the stimulus current increased, more fascicles were recruited. Therefore more action potentials added up to produce higher amplitude signals, and the electrodes 220 on the flexible neural ribbon electrodes 200b recorded larger signals. However, when the stimulus current increased to some extent (around 0.55 mA on this rat), all the fascicles were recruited, and the corresponding recorded amplitudes reached their thresholds, remaining constant in spite of further increases in the stimulus current.

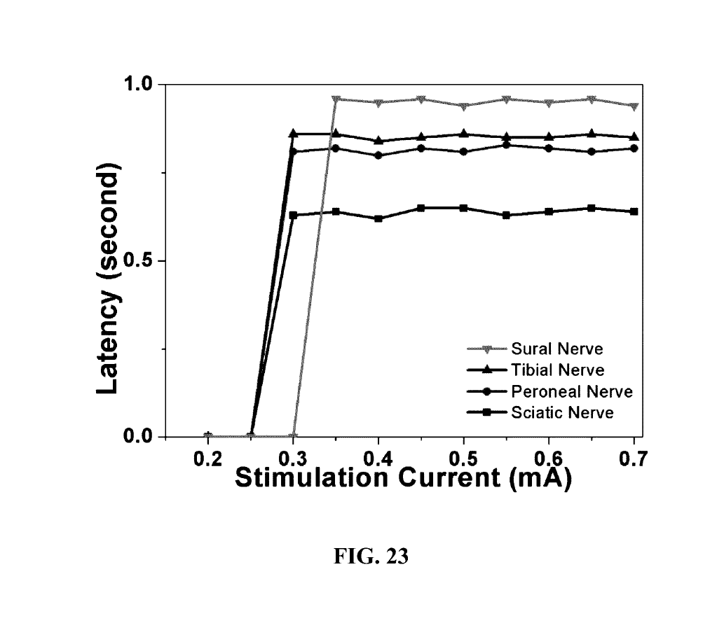

During an acute test, the latency value and the distance between the stimulating sites and recording electrodes 220 was also measured to calculate nerve conduction velocity. The latency for the measured CAP signal in each implanted neural ribbon was obtained under different stimulation conditions. Since the neural activity measured at Region 2 in FIG. 20B was most consistent for recordings, it was used for tracking latency on different neural ribbons over the experimental period. The results are shown in FIG. 23. The flexible neural ribbon electrode 200b that was implanted on the sciatic nerve was the closest to the stimulation hook electrodes. The signal latency was smaller than that recorded by flexible neural ribbon electrodes 200b attached on the branch terminals. However, when stimulated with different currents, all latencies in these flexible neural ribbon electrodes 200b remained almost constant. The conduction velocity of single fibers only depended on its diameter and the nerve bundles were composed of fibers of varying diameters. Fast fibers that had larger diameters contributed action potentials occurring towards the start of the CAP, while slower fibers with smaller diameters contributed action potentials found towards the tail section of the CAP. As long as the fibers in the nerve bundle were activated, the conduction velocity was fixed but the signal wave shape and duration may increase with increasing stimulation amplitude. Since the flexible neural ribbon electrode 200b that was implanted on the sciatic nerve was 3 cm away from the stimulation site, the conduction velocity was approximately 46 m/s.

### Compartment-Based Embedded Nerve Tissue—Electrode Interface Structures

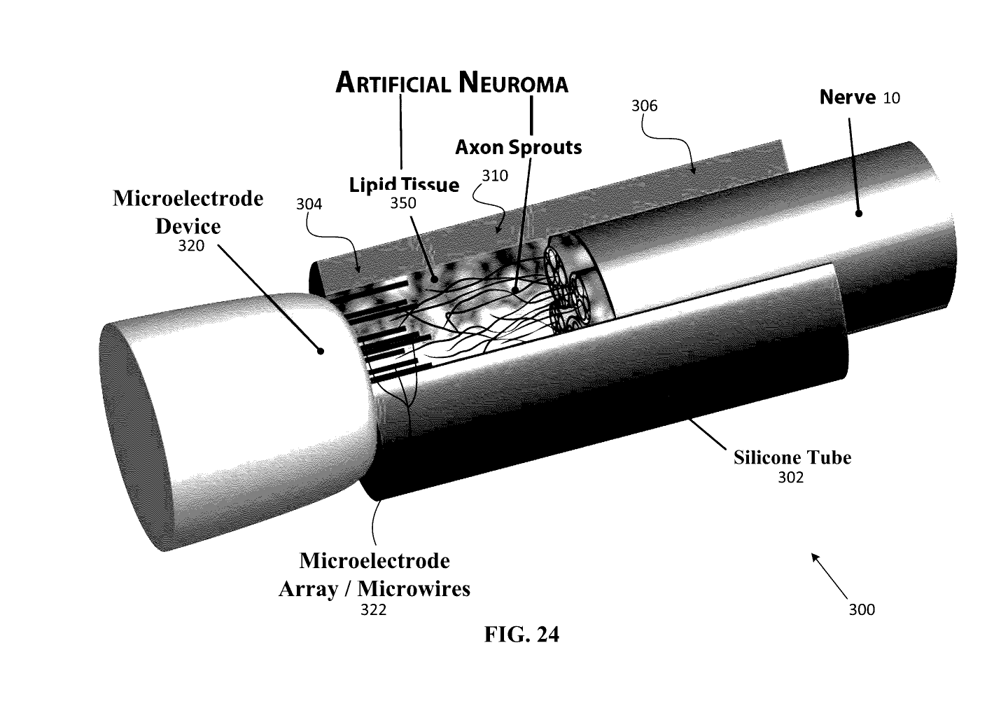

FIG. 24 is a schematic illustration of a compartment-based embedded nerve tissue—electrode interface structure 300 that facilitates or enables the generation of a self-organized nerve tissue—electrode interface in accordance with an embodiment of the present disclosure. In an embodiment, the embedded nerve tissue—electrode interface structure 300 includes a tube or tubular compartment or chamber 302 made of a biocompatible material (e.g., a biocompatible polymer such as Silicone). The tubular compartment 302 includes a first section or segment 304 at which portions of a microelectrode device 320 resides, such that electrical signal transfer structures or materials 322 provided by the microelectrode device 320 (e.g., an array of electrode microwires 322) resides, protrudes, or extends into portions of the compartment 302; a second section or segment 306 disposed opposite to the first segment 304, which includes an aperture or opening into which the terminal end of a transected peripheral nerve 10 can be inserted, such that the terminal end of the transected peripheral nerve faces toward the electrical signal transfer structures or materials 322 (e.g., the array of electrode microwires 322); and an intermediary region 310 between the first segment 304 and the second segment 306, into which the electrical signal transfer structures or materials 322 (e.g., microwires 322) extend, and in which autologous adipose tissue, growth-supportive cells or cellular populations (e.g., feeder glial cells, Schwann cells, and/or stem cells that can provide paracrine support for nerve growth), and possibly one or more types of nerve growth stimulant reside to provide a favourable medium for axonal sprouting and growth.

Spontaneous axonal sprouting within the intermediary region 310 results in the growth of a fibro-collagenous axonal tissue matrix in the intermediary region 310, and the formation of an end neuroma in the intermediary region 310 in the form of a self-organized nerve interface cone that surrounds and physically contacts the electrical signal transfer structures or materials 322 (e.g., one or more electrode microwires 322). The nerve interface cone includes axonal tissue therein that is capable of communicating or transferring neuroelectric signals to the electrical signal transfer structures or materials 322 (e.g., the electrode microwires 322).

In an experimental setup, the phenomenon of concurrent fibro-collagenous organization and axonal growth as a means for the creation of a functional interface with a conventional microelectrode device 320 was explored. The aim was to induce an inflammatory reaction interposed between the cut end of a peripheral nerve and a microelectrode array 322 (e.g., an array of electrode microwires 322), using autologous adipose tissue as a nerve growth stimulus. The fibro-proliferative response induced by the ischemic adipose tissue resulted in the self-organized growth of fibro-collagenous tissue around the microelectrode array 322, while trapping axonal fibers emerging from the terminal end of the transected nerve in the immediate vicinity of the fibro-collagenous tissue, resulting in the formation a neuroma-like structure around the microelectrode array 322.

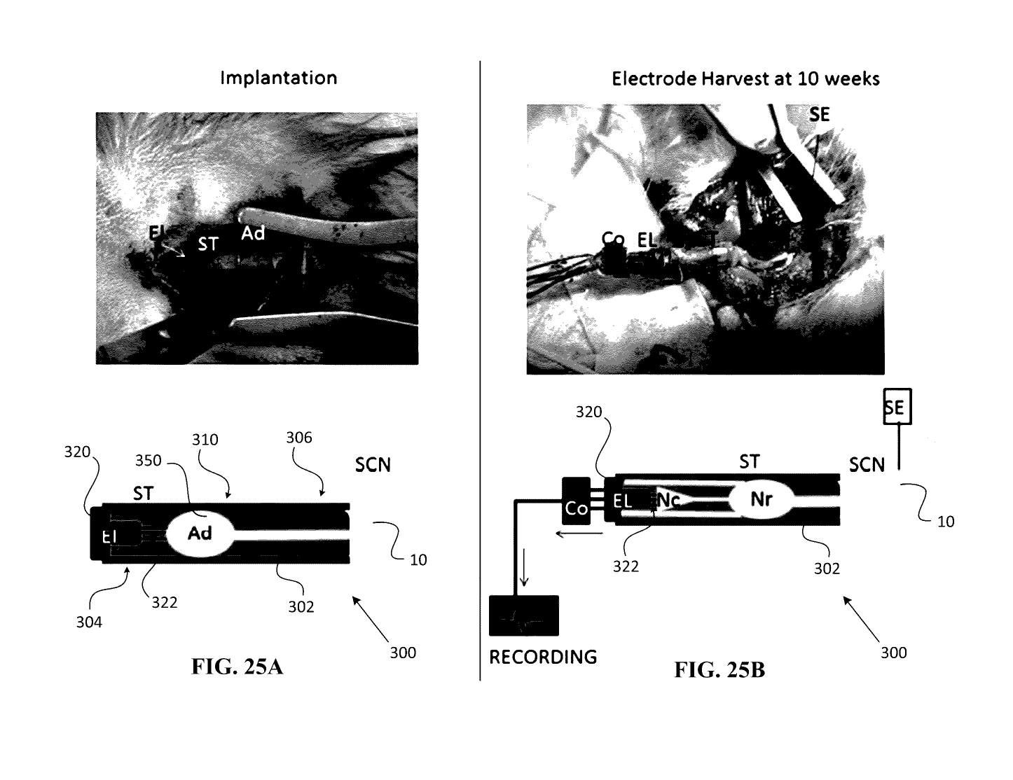

In this experiment, a 6-pin or 6-microwire tungsten microelectrode array 322 (Microprobe USA, tip size of 6 μm, 6 mm length) was incorporated at one end of a medical grade silicone tube 302 (1.5 cm long and 0.5 cm in diameter). The tube 302 was split longitudinally to allow placement of the nerve inside the tube 302. Five Sprague-Dawley female rats (weighing 300 to 400 grams) were operated on using intraperitoneal ketamine anesthesia under sterile surgical conditions. The left sciatic nerve was exposed through an incision immediately posterior and parallel to the femur. The sciatic nerve was identified and divided 0.5 cm proximal to the popliteal-fossa. The cut-end of the sciatic nerve was inserted into the microelectrode-tube assembly. A nidus of autogenous adipose tissue (4 to 5 mm diameter) was harvested from the popliteal region, and was placed in contact with the micro-electrodes 322 (interposed between the nerve end and the microelectrode array 320) ensuring that the end of the nerve was not in physical contact with the microelectrode array 320, as shown in FIG. 25A. The tube 302 was then placed in the intermuscular space posterior to the femur and anchored to the surrounding muscle. Fibrin glue was used to stabilize the nerve and the adipose graft within the tube 302, and the wound was closed. The rats were allowed unrestricted locomotion and were maintained on a normal diet for a period of 10 weeks.

At 10 weeks, the rats were anaesthetized, and the silicone tube 302 was delivered from the wound, maintaining the nerve (SCN) in situ, as shown in FIG. 25B. The microelectrode 320 (EL) was connected to a recording unit through a connector (Co), while a stimulating needle electrode (SE) was placed in contact with the sciatic nerve proximally.

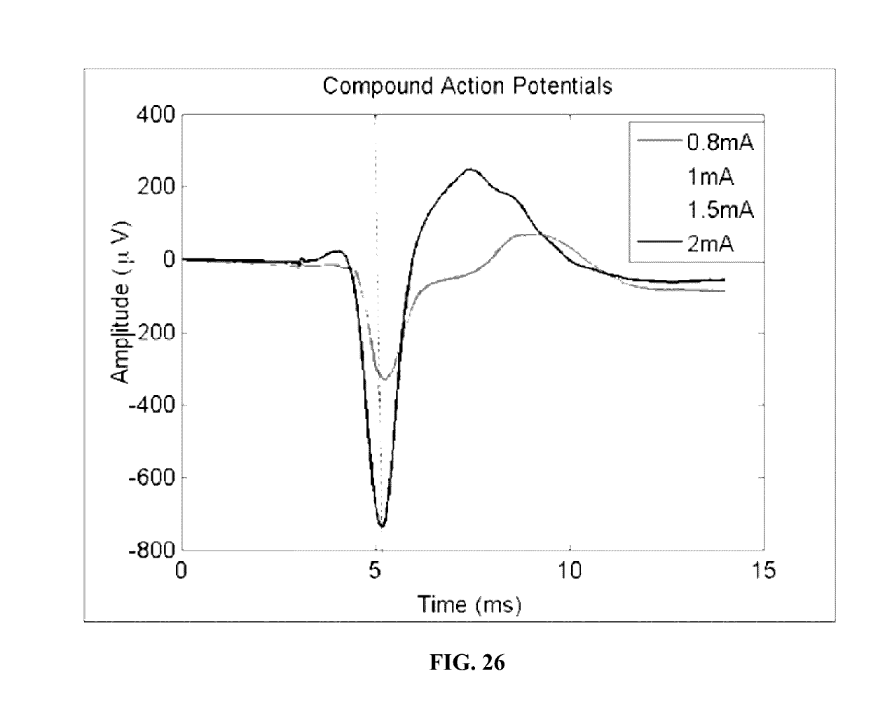

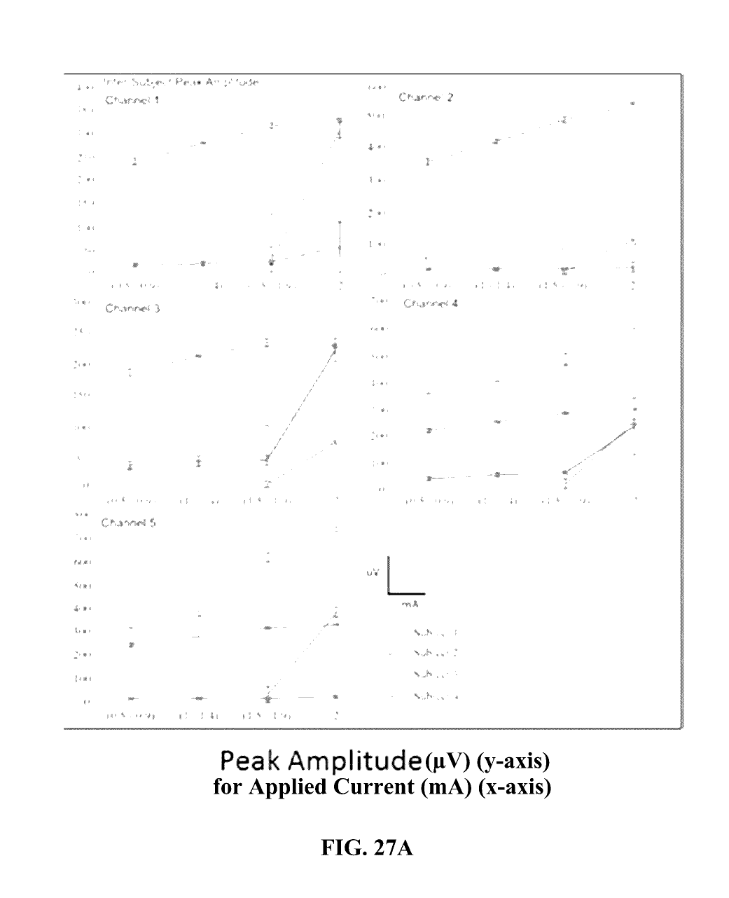

The microelectrode device 320 had 6 sensing electrodes in the form of (micro) pins or microwires 322. One of the 6 electrodes was selected as the reference electrode, hence a 5-channel recording was obtained in each study using a neural amplifier (Multi Channel Systems MCS GmBH, Reutlingen, Germany). The sciatic nerve was stimulated to evoke action potentials in the transected nerve. The stimulation paradigm consisted of monophasic current pulses of 20 μs duration, and amplitudes varying from 0.5 mA to 2 mA at 1 Hz frequency. Up to 50 raw compound action potential measurements were made for each combination of parameters used. FIG. 26 illustrates the average CAP of the repeated recordings made at varied levels of stimulation current. The distance between the site of stimulation and the recording electrode 322 was varied between 30 to 40 mm in each experiment, and this distance was used to calculate the nerve conduction velocity. The experiment was repeated in five subjects. Compound action potentials were observed and characterized in four out of the five rats studied. Electrode displacement in one of the subjects prevented recording of compound action potentials.

The raw traces obtained from all subjects were analyzed to identify the best point representation of the amplitude. The maximum peak recorded at 5 millisecond (ms) time point (dotted line) in FIG. 26 was used. This peak point was identified to be consistent across all recordings. The latency of this peak was used to calculate the nerve conduction velocity A minimum current in the range of 0.5-0.9 milli-ampere (mA) was required in order to evoke action potentials. An exception was observed in Subject 2 in that at stimulation current levels in the (0.5-0.9) mA and (1-1.4) mA range, the spike rate of the CAP was found to be 68.42% and 87.71% respectively. This was taken into consideration by ignoring the missed spike traces, while calculating mean and standard deviation values.

The electrodes 322 were harvested en-bloc and fixed in paraformaldehyde for 48 hours. A longitudinal window was created in the silicone tube 302 (ST) to examine the interior. Gross visual examination of the structures was carried out in-situ under 20× optical magnification. The structures observed within the tube were extracted without damage and subjected to standard paraffin histology (10 μm sections) for Haematoxylin and Eosin, as well as standardized protocol for immune-histochemical staining for the presence of Vimentin and Neurofilament markers. Photo-micrographic measurements were carried out using the reference scale built in within the microscope's digital imaging software.

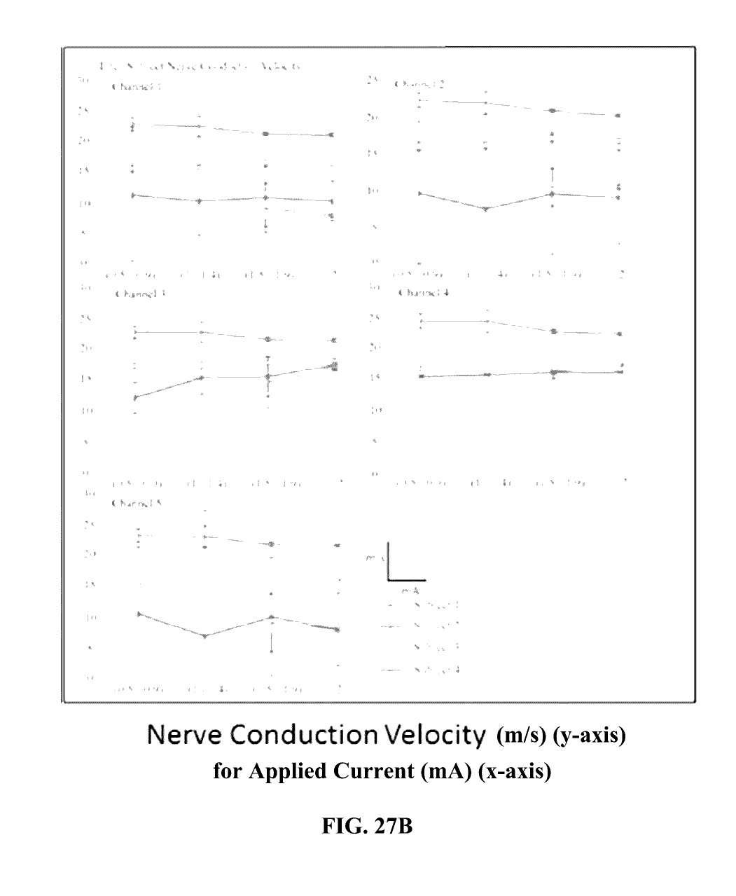

The plots in FIGS. 27A and 27B show the values of peak amplitude and nerve conduction velocity, respectively, on the Y-axis, recorded at each of the 5 sensing electrodes (Channels 1 to 5) for each subject recorded at increasing current intensity represented on the X-axis. The mean nerve conduction velocities across the interface showed variation between the four subjects. The maximum conduction velocity was seen in Subject #2, which had a range from 20.15±0.22 to 23.38±1.15 m/sec, and the minimum conduction velocity was noted in Subject #4, ranging from 6.99±20.49 to 17.67±1.00 m/sec. The peak amplitude was also variable between subjects. The highest value of 550.63±5.08 μV was seen in Subject #2 at 2 mA stimulation, while lower values were seen for Subjects #1 and #4, with ranges between 10.76±5.46 μV and 386.71±21.42 μV (FIG. 4).

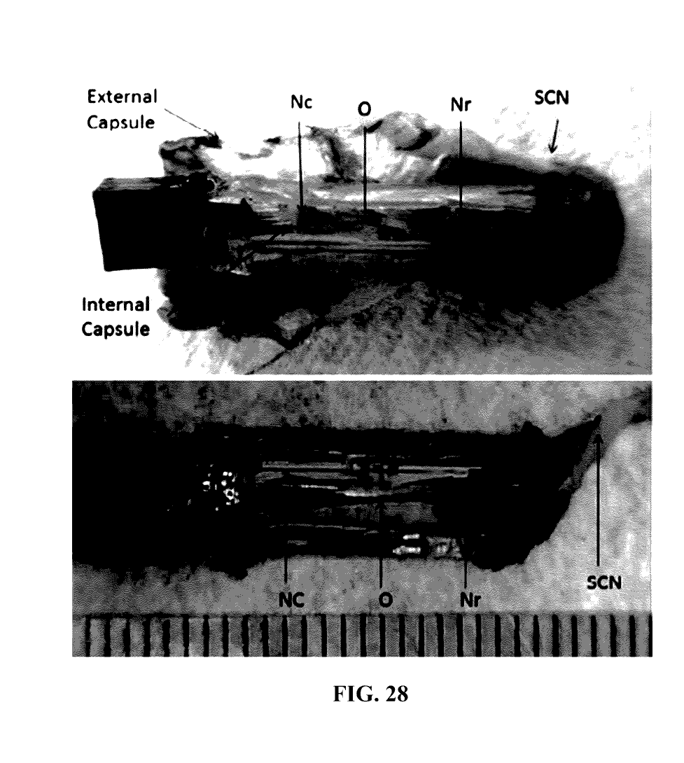

The entire implant was found to be encased in a fibrous capsule (external capsule). The capsule was split, and internal structures were examined in situ, as shown in FIG. 28. A uniform fibro-collagenous internal capsule (internal capsule) lining the inner surface of the silicone tube 302, which was structurally in continuity with the proximal end of the nerve as well as the electrodes 322 was also noted and harvested for histology.

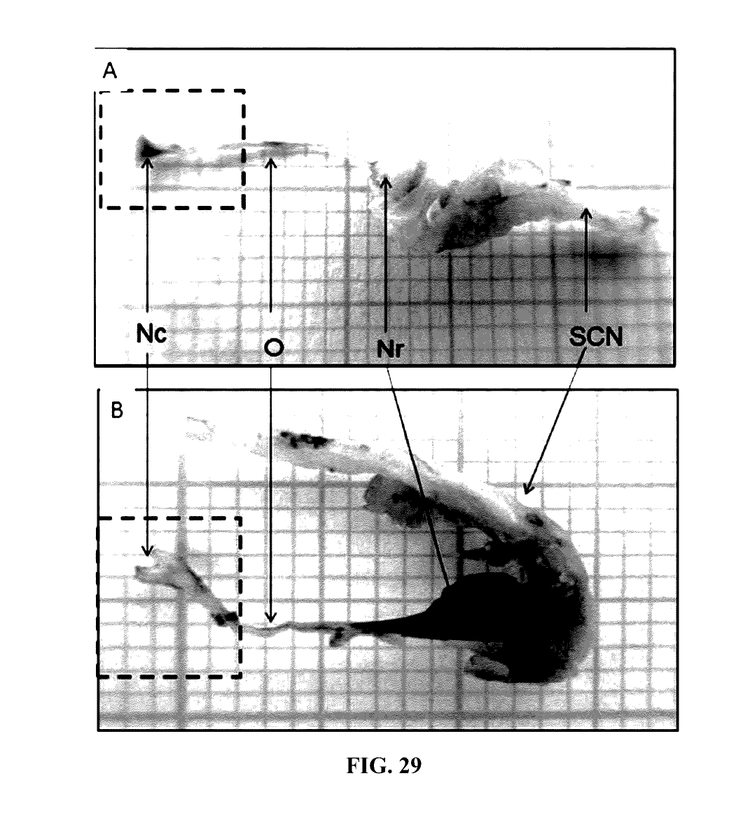

Examination under magnification as shown in FIGS. 28 and 29 showed encasement of the microelectrodes within a well-defined cone-like structure in Subjects #2 and #3, which can be termed the ‘nerve interface cone’ (Nc). The microelectrode tips were encased by this tissue over a length of 4 mm. A magnified view is shown in FIG. 29. The nerve interface cone was found to be in continuity with the terminal end (Nr) of the cut sciatic nerve (SCN), through a 0.3 mm to 0.5 mm wide bridge segment (0) (FIG. 5). In Subjects #1 and #4, a less well-defined or partial organization of nerve interface cone around the tips was seen.

The tissues were extracted from the implant without damage and a consistent morphology was observed between the specimens. The sciatic nerve was seen to end in a bulbous structure (Nr), which gave rise to a bridge segment (O) that then ended in the formation of the nerve interface cone (Nc). A magnified view of the spontaneously formed ‘nerve interface cone’ (Nc) shows a well-defined architecture as seen against a 1 mm grid in FIG. 29.

It is of note that the ‘nerve interface cone’ (Nc) and the bridge segment (0) were formed as a result of the biological self-organization of structural growth from the terminal end of the nerve (Nr), without any physical manipulation. The similar morphology in these self-organized structures across the subjects indicates that this model can be replicated.

Immunohistochemistry of the wall of the nerve interface cone showed that the nerve-cone was predominantly composed of a layered composite of fibro-collagenous layer ranging from 20 μm to 100 μm in thickness, with a single layer of linear Neurofilament positive fibers 10 μm in thickness. The Neurofilament-positive axonal layer was sandwiched between the Vimentin-positive fibroblastic layers. No remnant of adipose tissue was observed in the sections. The distance of the axonal layer to the surface of the composite tissue was in the range of 10 μm to 50 μm.

Immunohistochemistry of the capsule lining the inner surface of the silicone tube 302 (internal capsule) was also found to contain a layered arrangement of Vimentin-positive cells (fibroblasts) 200 μm in thickness, and a layered arrangement of Neurofilament-positive axonal fibers in all the subjects. The axonal layer in these specimens was also found to be sandwiched between the Vimentin-positive fibroblast layers, which were 100 μm thick at the origin, but tapered to thicknesses of 10 μm distally. Terminal ends of the growing axons were also noted in this layer. In all regions examined, the Neurofilament-stain positive layer followed the contours of the fibroblastic layer.

The formation of an organized fibro-collagenous capsule is a well-documented phenomenon around silicone as well as metallic implants. The initial stage of reactive exudate formation on the material surfaces is followed by the organization of the exudate into a stable capsule composed of fibroblasts and collagen fibers. Based on observations, we infer that the inflammatory response to the silicone and metallic surfaces resulted in a stable fibro-collagenous tissue encapsulation of the micro-electrodes 322 and the silicone tube 302.

In this study, the process of axonal growth from the cut end of the nerve within the spatial constraint of the tube 302, which occurred synchronously with the process of capsular organization, resulted in the integration of axons within the fibro-collagenous structure. The well-organized laminar arrangement of axons within layers of the fibroblastic tissue points to a possible role of fibroblasts or collagen in providing substrate-guidance for the growing axons. This phenomenon of self-organization of a layered integration of axons within a fibro-collagenous tissue has not been previously described in the literature. The growth of the self-organizing fibro-collagenous structure and the axonal tissue therein can be influenced by the structure of the device 300, for instance, based upon device shape, dimensions/size, materials construction, internal medium content, and the arrangement or organization of electrical signal transfer structures or materials within the device 300.

Without wishing to be bound by a specific theory, the role of adipose tissue as an initiator of inflammation may have produced a localized inflammatory process that resulted in exudate formation in the space between the nerve end and the electrode resulting in the organization of the ‘nerve interface cone’ and a bridging segment in the empty space in the axis of the tube 302 in the total absence of any physical substrate. The electrophysiological characteristics of this composite tissue differ from a normal nerve. The conduction velocities across the newly formed interface showed variability between the subjects Although the values were different for individual subjects, they were consistent for each subject across different stimulation currents and electrodes. Maximum values for nerve conduction velocity observed across the fibro-axonal interface was 23.38±1.15 m/sec compared to reported velocities in the range of 40 to 50 m/sec in normal sciatic nerves in rats. This might be related to the presence of fibro-collagenous tissue as a part of the interface. The axonal layer was separated from the microelectrode 322 by a fibroblastic layer, which varied from 10 μm to 50 μm in different regions. The electrophysiological conduction was achieved through axonal proximity to the electrode rather than direct contact. Based on these observations, it is possible that the electrophysiological properties may not degenerate over time once collagen maturation has been achieved around the electrodes with the formation of a stable fibro-collagenous layer, with no further progression of fibrosis.

This embedded neural tissue—electrode interface relies on the integration of the two biological processes of fibroblastic organization and axonal growth, and requires a period of maturation before functional signals can be recorded. Due to the inherent nature of the fibro-collagenous tissue, the structure may be likely to maintain stable proximity for the axonal content to the electrodes.

Aspects of particular embodiments of the present disclosure address at least one aspect, problem, limitation, and/or disadvantage associated with exiting neural electrode structures and devices. While features, aspects, and/or advantages associated with certain embodiments have been described in the disclosure, other embodiments may also exhibit such features, aspects, and/or advantages, and not all embodiments need necessarily exhibit such features, aspects, and/or advantages to fall within the scope of the disclosure. It will be appreciated by a person of ordinary skill in the art that several of the above-disclosed systems, components, processes, or alternatives thereof, may be desirably combined into other different systems, components, processes, and/or applications. In addition, various modifications, alterations, and/or improvements may be made to various embodiments that are disclosed by a person of ordinary skill in the art within the scope and spirit of the present disclosure.

1. A flexible epineural strip electrode for a peripheral nerve, comprising: a single flexible substrate having a nerve interface portion and an electronics interface portion that extends away from the nerve interface portion, wherein the nerve interface portion includes an inner surface configured for direct placement upon the epineurium of the peripheral nerve, wherein the inner surface carries a set of exposed electrodes configured for contacting the epineurium of the peripheral nerve, wherein the electronics interface portion carries at least one set of electrical pads to which an electrical device distinct from the flexible substrate can be electrically coupled, and wherein the nerve interface portion and the electronics interface portion carry integrated circuit wiring by which the set of electrical contacts is electrically coupled to at least one set of electrical pads.

2. The flexible epineural strip electrode of claim 1, wherein the nerve interface portion includes a plurality of suture apertures formed therein by which the nerve interface portion is suturable to the peripheral nerve, another anatomical structure, or itself.

3. The flexible epineural strip electrode of claim 1, further comprising an integrated circuit chip, a flexible printed circuit (FPC), or a flexible flat cable (FFC) bonded to the at least one set of electrical pads, wherein the integrated circuit chip, the FPC, or the FFC corresponds to a neural amplifier or a neural stimulator.

4. The flexible epineural strip electrode of claim 1, wherein the flexible substrate comprises polyimide or parylene.

5. The flexible epineural strip electrode of claim 1, wherein the nerve interface portion comprises at least one flexible elongate strip.

6. The flexible epineural strip electrode of claim 5, wherein the nerve interface portion comprises a plurality of flexible elongate strips disposed in a parallel arrangement with respect to each other, wherein each flexible elongate strip includes an inner surface configured for direct placement on the epineurium of the peripheral nerve, and wherein the inner surface of each flexible elongate strip carries a plurality of exposed electrodes configured for contacting the epineurium of the peripheral nerve.

7. A flexible epineural strip electrode for a peripheral nerve, comprising: a single flexible substrate having a front side configured for facing away from the epineurium of the peripheral nerve and a back side configured for direct placement upon the epineurium of the peripheral nerve, wherein the front side carries a neural amplifier or neural stimulator, and wherein the back side carries a plurality of exposed electrodes configured for contacting the epineurium of the peripheral nerve.

8. The flexible epineural strip electrode of claim 7, wherein the flexible substrate includes a plurality of suture apertures formed therein by which the flexible substrate is suturable to the peripheral nerve, another anatomical structure, or itself.

9. The flexible epineural strip electrode of claim 7, wherein the flexible substrate comprises polyimide or parylene.

10. The flexible epineural strip electrode of claim 7, wherein the single flexible substrate comprises a plurality of flexible strips, each flexible strip having a front side configured for facing away from the epineurium of the peripheral nerve and a back side configured for direct placement upon the epineurium of the peripheral nerve, wherein the plurality of flexible strips includes a first flexible strip that carries the neural amplifier or neural stimulator on its front side and which further carries a first set of exposed electrodes on its back side, and a second flexible strip that carries a second set of exposed electrodes on its back side.

11. The flexible epineural strip electrode of claim 10, wherein each flexible strip is structurally coupled to an adjacent flexible strip by way of a set of arm members, and wherein each flexible strip includes suture apertures formed therein by which the flexible strip is sutrable to the peripheral nerve, another anatomical structure, itself, or another flexible strip.

12. A flexible neural ribbon electrode for a peripheral nerve, comprising a single flexible substrate having:
an elongate ribbon section having an outer surface configured for facing away from the epineurium of the peripheral nerve and an inner surface configured for facing toward the epineurium of the peripheral nerve, wherein the elongate ribbon section is spirally windable about the epineurium along a portion of a length of the peripheral nerve;
a plurality of electrodes disposed along and projecting from the inner surface of the elongate ribbon section; and
a first end portion providing a connection pad structure having a plurality of electrical pads to which an electronic device distinct from the flexible neural ribbon electrode is electrically couplable or bondable.

13. The flexible neural ribbon electrode of claim 12, further comprising a second end portion, wherein the elongate ribbon section extends between the first end portion and the second end portion.

14. The flexible neural ribbon electrode of claim 13, wherein the first end portion and the second end portion include suture apertures formed therein by which the first end portion and the second end portion, respectively, are suturable to the peripheral nerve, one or more other anatomical structures, and/or themselves.

15. The flexible neural ribbon electrode of claim 12, wherein the plurality of electrodes comprise microneedle electrodes configured for penetrating the epineurium, and/or stud type electrodes configured for directly residing upon the epineurium surface.

17. The flexible neural ribbon electrode of claim 15, further comprising a reference electrode carried by an inner surface of the flexible neural electrode.

18. The flexible neural ribbon electrode of claim 12, wherein the flexible substrate comprises polyimide or parylene.

19. An embedded nerve tissue—electrode interface structure, comprising:
a biocompatible tubular compartment having a first segment, a second segment disposed opposite to the first segment, and an intermediary region that extends between the first segment and the second segment;
a microelectrode device having a set of electrical signal transfer structures disposed at the first segment of the tubular compartment, which extend into the intermediary region;
an aperture within the second segment configured for receiving a severed peripheral nerve such that a terminal end of the peripheral nerve is disposed in the intermediary region and faces the set of electrical signal transfer structures; and
a medium that promotes axonal cellular growth carried within the intermediary region.

20. The embedded nerve tissue—electrode interface of claim 19, wherein the tubular compartment comprises silicone.

21. The embedded nerve tissue—electrode interface of claim 19, wherein the medium comprises at least one of autologous adipose tissue, glial cells, Schwann cells, stem cells, and a nerve growth stimulant.

22. The embedded nerve tissue—electrode interface of claim 19, wherein the set of electrical signal transfer structures comprises an array of microwires.

23. The embedded nerve tissue—electrode interface of claim 19, further comprising a self-organized nerve interface cone comprising fibro-collagenous axonal tissue that surrounds and physically contacts the set of electrical signal transfer structures.
